(19) United States
(12) Patent Application Publication
(10) Pub. No.: US 2024/0279285 A1
(43) Pub. Date: Aug. 22, 2024

(54) Rippled Antiparallel Cross-Beta Dimers and Related Materials, Compositions and Methods

(71) Applicant: The Regents of the University of California, Oakland, CA (US)

(72) Inventor: Jevgenij A. Raskatov, Santa Cruz, CA (US)

(21) Appl. No.: 18/570,390

(22) PCT Filed: Jun. 17, 2022
(86) PCT No.: PCT/US2022/034076

§ 371 Date: Dec. 14, 2023

Related U.S. Application Data
(60) Provisional application No. 63/211,980, filed on Jun. 17, 2021.

(51) Int. Cl. C07K 14/00

(52) U.S. Cl. CPC C07K 14/001

(57) ABSTRACT

Provided are rippled antiparallel cross-β dimers. In some embodiments, the dimers comprise (L,L,L)-(FX₁F)ₖ dimerized with (D,D,D)-(FX₂F)ₖ, where X₁ and X₂ are independently selected from any amino acid, and wherein k is an integer of 1 or greater. Also provided are rippled β-sheet fibrils comprising a plurality of the rippled antiparallel cross-β dimers of the present disclosure. Materials comprising the rippled antiparallel cross-β dimers and rippled β-sheet fibrils of the present disclosure are also provided, as are compositions comprising such materials. Also provided are methods of making the rippled antiparallel cross-β dimers and rippled β-sheet fibrils of the present disclosure.

### CROSS-REFERENCE TO RELATED APPLICATIONS

This application claims the benefit of U.S. Provisional Patent Application No. 63/211,980, filed Jun. 17, 2021, which application is incorporated herein by reference in its entirety.

### STATEMENT OF GOVERNMENT SUPPORT

This invention was made with Government support under Grant Nos. F31AG066377, R21AG058074, and R21AG070888, awarded by the National Institutes of Health (NIH); Grant No. 2018501 awarded by the National Science Foundation. The Government has certain rights in the invention.

### INTRODUCTION

Peptides with mixed chirality may be used to access frameworks with unique properties, including protease-resistant peptide drugs,^{1,2} hydrogels with enhanced rigidity,^{3,4} aggregation blockers,^{5,6} amyloid oligomer-to-fibril converters,^{7,8} and mechanistic tools.^{9,10} Mirror-image proteins may also be used to enhance crystallization of proteins that are hard to crystallize, sometimes by creating unique interactions between the protein enantiomers.¹¹⁻¹⁴ A systematic incorporation of D-amino acids into proteins and peptides is expected to give access to a huge structure-function space that cannot be accessed in any other way.

In 1951, Pauling and Corey introduced the pleated β-sheet as a two-dimensional periodic layer configuration built from extended homochiral peptide strands.¹⁵ The pleated β-sheet rapidly established itself as a key protein structural motif that is commonly known in textbooks as the β-sheet. Thousands of protein structures have been published that contain β-sheets. This includes structures that may be as huge as a periodic, fibrillary β-sheet network on the one side and as small as a β-sheet dimer in the context of a globular protein on the other side. In 1953, Pauling and Corey introduced the rippled β-sheet as a configuration closely related to the pleated β-sheet, but with every alternate peptide chain mirrored, thus giving rise to unique structures.¹⁶ Some of the key structural differences between pleated and rippled β-sheets, including differences in hydrogen bonding and relative side-chain disposition in the β-sheet frameworks, have been discussed very recently.¹⁷ As illustrated in FIG. 1, in an antiparallel pleated sheet, amino acid side chains are aligned in a vertical line orthogonal to the peptide backbones (FIG. 1, left panel). In contrast, in an antiparallel rippled sheet, to reduce steric repulsion between the alternating enantiomeric peptides, the side chains are oriented diagonally across the peptidic network (FIG. 1, right panel).

Unlike with the pleated β-sheet (now known as the β-sheet), the growth of the present body of knowledge on the rippled β-sheet has been extremely sluggish. The first experimental observation of an (antiparallel) rippled sheet was made in the 1970s by Lotz, Moore and Krimm, on polyglycine I.¹⁸⁻²⁰ The authors used space group considerations to conclude that polyglycine I crystals contained rippled rather than pleated antiparallel sheets (monoclinic rather than orthorhombic unit cell geometry). Some three decades later, Lahav and co-workers used clever labeling strategies in conjunction with mass-spectrometry, to produce evidence for rippled sheet formation, based on templated peptide replication.^{21,22} Conversely, Chung and Nowick noted in their solution-phase NMR studies a thermodynamic preference for a dimeric pleated β-sheet, with the alternative rippled sheet observed as a minor diastereomer.²³ A more recent study by Liu and Gellman is broadly consistent with Chung and Nowick.²⁴ The present understanding of the interplay of thermodynamics and kinetics that underlie the formation of pleated vs rippled sheets remains extremely limited. Experiments performed in the laboratories of Schneider,^{3,4} Nilsson,^{25,26} Raskatov,^{7,8} and Torbeev,²⁷ showed that mirror-image peptide strands may assemble into rippled sheets, but there is also evidence that some sequences may favor homochiral association.²⁸ The structural insights available for the MAX1/DMAX systems,³ a short Amyloid-β (Aβ) segment,²⁵ and, most recently, racemic full-length Aβ40²⁹ were obtained from theoretical calculations constrained by a fairly limited number of experimental data. These studies provide valuable insights into rippled sheets, but not experimental high-resolution structures.

Not all racemic peptide mixtures form rippled sheets,^{23,24,30} as self-sorting into pleated sheets may also occur.³⁰ The field is just beginning to learn why some racemic peptide mixtures form rippled sheets (i.e., are “ripple-genic”), whereas others prefer to form pleated sheets instead (i.e., are “pleat-genic”). To systematically map out the structure-function space and to close this major knowledge gap, high-resolution structures of rippled sheets are needed.

### SUMMARY

Provided are rippled antiparallel cross-β dimers. In some embodiments, the dimers comprise (L,L,L)-(FX₁F)ₖ dimerized with (D,D,D)-(FX₂F)ₖ, where X₁ and X₂ are independently selected from any amino acid, and wherein k is an integer of 1 or greater. Also provided are rippled β-sheet fibrils comprising a plurality of the rippled antiparallel cross-β dimers of the present disclosure. Materials comprising the rippled antiparallel cross-β dimers and rippled β-sheet fibrils of the present disclosure are also provided, as are compositions comprising such materials. Also provided are methods of making the rippled antiparallel cross-β dimers and rippled β-sheet fibrils of the present disclosure.

### BRIEF DESCRIPTION OF THE FIGURES

FIG. 1: Left panel: Antiparallel pleated sheet in different projections. Right panel: Antiparallel rippled sheet in different projections. A selected number of amino acid side chains are depicted as large spheres on the left panel (pleated, along vertical arrow) and on the right (rippled, along diagonal arrow), to reduce steric repulsion in each case.

FIG. 2: Ball-and-stick depiction of the experimental rippled antiparallel FFF:fff cross-β dimer, shown in three orthogonal projections. The Pauling-Corey rippled antiparallel backbone dimer is shown in the inset, with apical carbon atoms added geometrically to facilitate comparison.

FIG. 3: Long-range packing of the FFF:fff lattice, shown in three orthogonal projections. The layer-to-layer distance is indicated.

FIG. 4: A top-on view of a single layer containing the peptidic backbones. Individual rippled antiparallel FFF:fff cross-β dimers are centered about the unit cell corners and center.

FIG. 5: Detail of the antiparallel rippled motifs in the proteins selected by the PDB structural database mining. (A) glu-lys-glu-leu-val sequence in RV1738.⁴² (B) phe-phe-tyr sequence in ester insulin.⁴³ (C) lys-gly-phe-arg sequence in Kaliotoxin.⁴⁴ PDB codes are displayed on the bottom right.

FIG. 6: Ramachandran angle analysis for the rippled sheets noted with A) the FFF:fff system; B) racemic Ester Insulin (4IUZ)⁴³; C) racemic RV1738 (4WPY)⁴²; D) racemic Kaliotoxin (3ODV).⁴⁴

FIG. 7: Pleated and rippled antiparallel periodic β-sheet layers hypothesized by Pauling and Corey. C_{β} carbons are shown as black spheres; C_{β} carbons are shown as spheres and those above the plane of the sheet are circled for clarity.

FIG. 8A-8C: The periodic rippled antiparallel β-sheet [FYF:fyf]n layer, shown in two orthogonal projections (A,B). Dihedral angles in [°]; H-bond distances in [Å]; (L,L,L)-tripeptides and (D,D,D)-tripeptides are shown. Packing in the crystallographic lattice; four symmetry-equivalent columns shown (C).

FIG. 9A-9C: The periodic rippled antiparallel β-sheet [FWF:fwf]n layer, shown in two orthogonal projections (A,B). Dihedral angles in [°]; H-bond distances in [Å]; (L,L,L)-tripeptides and (D,D,D)-tripeptides are shown. Packing in the crystallographic lattice; four symmetry-equivalent columns shown (C).

FIG. 10A-10C: The periodic rippled antiparallel β-sheet [FWF:fyf]n layer, shown in two orthogonal projections (A,B). Dihedral angles in [°]; H-bond distances in [Å]; (L,L,L)-tripeptides and (D,D,D)-tripeptides are shown. Packing in the crystallographic lattice; four symmetry-equivalent columns shown (C).

FIG. 11A-11C: The periodic pleated parallel β-sheet [FYF:FYF]n layer, shown in two orthogonal projections (A,B). Dihedral angles in [°]; H-bond distances in [Å]. Packing in the crystallographic lattice; four symmetry-equivalent columns shown (C).

FIG. 12A-12C: Illustrations of the crystal structure of FWF:fyf.

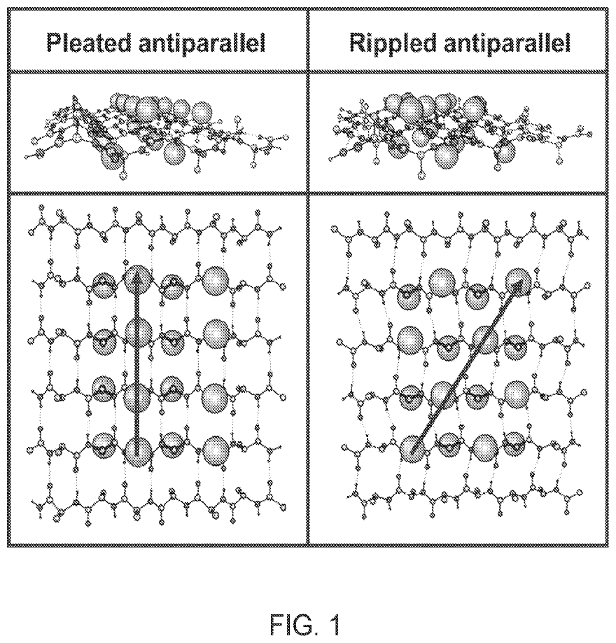

### DETAILED DESCRIPTION

Before the dimers, fibrils and methods of the present disclosure are described in greater detail, it is to be understood that the dimers, fibrils and methods are not limited to particular embodiments described, as such may, of course, vary. It is also to be understood that the terminology used herein is for the purpose of describing particular embodiments only, and is not intended to be limiting, since the scope of the dimers, fibrils and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the dimers, fibrils and methods. The upper and lower limits of these smaller ranges may independently be included in the smaller ranges and are also encompassed within the dimers, fibrils and methods, subject to any specifically excluded limit in the stated range. Where the stated range includes one or both of the limits, ranges excluding either or both of those included limits are also included in the dimers, fibrils and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the dimers, fibrils and methods belong. Although any dimers, fibrils and methods similar or equivalent to those described herein can also be used in the practice or testing of the dimers, fibrils and methods, representative illustrative dimers, fibrils and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present dimers, fibrils and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the dimers, fibrils and methods, which are, for clarity, described in the context of separate embodiments, may also be provided in combination in a single embodiment. Conversely, various features of the dimers, fibrils and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present dimers, fibrils and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Rippled Antiparallel Cross-B Dimers, Rippled B-Sheet Fibrils and Related Materials and Compositions

Aspects of the present disclosure include rippled antiparallel cross-β dimers. The rippled antiparallel cross-β dimers comprise a polymer of two or more L-amino acids dimerized with a polymer of two or more D-amino acids. The term “amino acid” generally refers to any monomer unit that comprises a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Other representative amino acids include, but are not limited to, amino acids comprising photoactivatable cross-linkers, metal binding amino acids, spin-labeled amino acids, fluorescent amino acids, metal-containing amino acids, amino acids with novel functional groups, amino acids that covalently or noncovalently interact with other molecules, photocaged and/or photoisomerizable amino acids, radioactive amino acids, amino acids comprising biotin or a biotin analog, glycosylated amino acids, other carbohydrate modified amino acids, amino acids comprising polyethylene glycol or polyether, heavy atom substituted amino acids, chemically cleavable and/or photocleavable amino acids, carbon-linked sugar-containing amino acids, redox-active amino acids, amino thioacid containing amino acids, and amino acids comprising one or more toxic moieties.

The term “amino acid” includes, but is not limited to, naturally-occurring α-amino acids and their stereoisomers. “Stereoisomers” of amino acids refer to mirror image isomers of the amino acids, such as L-amino acids or D-amino acids. For example, a stereoisomer of a naturally-occurring amino acid refers to the mirror image isomer of the naturally-occurring amino acid (i.e., the D-amino acid).

Naturally-occurring α-amino acids are those encoded by the genetic code as well as those amino acids that are later modified (e.g., hydroxyproline, γ-carboxyglutamate, and O-phosphoserine). Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (lie), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Commission on Biochemical Nomenclature. For example, an L-amino acid may be represented herein by its commonly known three letter symbol (e.g., Arg for L-arginine) or by an upper-case one-letter amino acid symbol (e.g., R for L-arginine). A D-amino acid may be represented herein by its commonly known three letter symbol (e.g., D-Arg for D-arginine) or by a lower-case one-letter amino acid symbol (e.g., r for D-arginine).

The terms “polypeptide,” “peptide,” “protein”, and “polymer of amino acids,” used interchangeably herein, refer to a polymeric form of amino acids of any length (e.g., connected one to the other by peptide bonds between the alpha-amino and carboxy groups of adjacent residues), which can include genetically coded and non-genetically coded amino acids, chemically or biochemically modified or derivatized amino acids, and polypeptides having modified peptide backbones.

In certain embodiments, the rippled antiparallel cross-β dimers comprise (L,L,L)-(FX₁F)ₖ dimerized with (D,D,D)-(FX₂F)ₖ, where X₁ and X₂ are independently selected from any amino acid, and where k is an integer of 1 or greater. “L” refers to levorotatory, “F” is phenylalanine, and “D” refers to dextrorotatory. According to some embodiments, k is an integer of from 1 to 1000, such as from 1 to 750, from 1 to 500, from 1 to 250, from 1 to 100, from 1 to 75, from 1 to 50, from 1 to 40, from 1 to 30, from 1 to 20, or from 1 to 10.

According to some embodiments, X₁ and X₂ are independently selected from the group consisting of: F, Y (tyrosine), and W (tryptophan). In certain embodiments, X₁ and X₂ are the same. That is X₁ and X₂ may be enantiomers of the same amino acid. In other embodiments, X₁ and X₂ are different, i.e., X₁ may be the L form of a first amino acid while X₂ is the D form of a second, different amino acid.

In certain embodiments, the dimers are water-soluble, i.e., capable of being dissolved in water. According to some embodiments, the termini of the monomers of the dimers comprise a moiety that renders the dimers water-soluble. Examples of such moieties include, but are not limited to, a free amine, a free carboxylate, and/or the like. By way of example, the N-terminus and/or C-terminus of the (L,L,L)-(FX₁F)ₖ monomer may comprise a free amine or a free carboxylate, thereby rendering the dimer water soluble. Also by way of example, the N-terminus and/or C-terminus of the (D,D,D)-(FX₂F)ₖ monomer may comprise a free amine or a free carboxylate, thereby rendering the dimer water soluble.

Also provided by the present disclosure are rippled β-sheet fibrils. The rippled β-sheet fibrils comprise a plurality of the rippled antiparallel cross-β dimers of the present disclosure.

Aspects of the present disclosure further include materials comprising a plurality of the rippled antiparallel cross-β dimers of the present disclosure. According to some embodiments, provided are materials comprising the rippled β-sheet fibrils of the present disclosure. In certain embodiments, such materials comprise the plurality of rippled antiparallel cross-β dimers held together by a combination of interdimer hydrogen bonds, ionic interactions, van der Waals interactions, or any combination thereof. For example, the materials may comprise the plurality of rippled antiparallel cross-β dimers held together by a combination of interdimer hydrogen bonds, ionic interactions, and van der Waals interactions.

Also provided are compositions comprising the materials of the present disclosure. In certain embodiments, such a composition includes a material of the present disclosure present in a liquid medium, e.g., an aqueous liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a protease inhibitor, glycerol, a chelating agent, and the like may be present in such compositions.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the materials against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included, e.g., in an amount of about 10 mM to 500 nM.

In certain embodiments, a composition of the present disclosure comprises the material and is essentially free of one or more preservatives, such as ethanol, benzyl alcohol, phenol, m-cresol, α-chlor-m-cresol, methyl or propyl parabens, benzalkonium chloride, and combinations thereof. In other embodiments, a preservative is included in the composition, e.g., at concentrations ranging from about 0.001 to about 2% weight/volume (w/v).

### Methods

Also provided by the present disclosure are methods of making and using the rippled antiparallel cross-β dimers, rippled β-sheet fibrils, materials and compositions of the present disclosure.

In certain embodiments, provided are methods comprising producing a polypeptide comprising, consisting essentially of, or consisting of, (L,L,L)-(FX₁F)ₖ, and producing a polypeptide comprising, consisting essentially of, or consisting of, (D,D,D)-(FX₂F)ₖ. Examples of such (L,L,L)-(FX₁F)ₖ and (D,D,D)-(FX₂F)ₖ polypeptides which may be produced include those described above and in the Experimental section below. According to some embodiments, the polypeptides are produced by chemical synthesis. A non-limiting example of a chemical synthesis includes solid-phase polypeptide synthesis. Solid-phase peptide synthesis (SPPS) involves the successive addition of protected amino acid derivatives to a growing peptide chain immobilized on a solid phase, including deprotection and washing steps to remove unreacted groups and also side products. Any number of solid supports may be employed, including resins such as polystyrene and polyamide based resins. The peptides may be covalently bound to a solid support, typically at their C-terminal end through linkers such as acid labile and photolabile linkers. In some embodiments the linker is an acid labile linker. In other embodiments, the linker is a trityl linker such as a 2-chlorotrityl linker. Peptide synthesis is typically performed by coupling a protected amino acid to the N-terminal end of the bound sample. The protected amino acid may contain N-terminal protecting groups such as a Boc (tert-butyloxycarbonyl) or Fmoc (9-fluorenylmethyloxycarbonyl) group as well as side chain protecting groups. According to some embodiments, when solid-phase polypeptide synthesis is performed to produce the polypeptides, the solid-phase synthesis is Fmoc-based solid-phase synthesis.

The above-described methods may further comprise purifying the produced (L,L,L)-(FX₁F)ₖ and (D,D,D)-(FX₂F)ₖ polypeptides. Any suitable approach for purifying the polypeptides may be employed. In certain embodiments, the polypeptides are purified by chromatography, a non-limiting example of which is High Performance Liquid Chromatography (HPLC). An example HPLC-based approach suitable for purifying the polypeptides is described in Warner et al. (2017) JoVE, 2017, e55482.

The above-described methods may further comprise combining the produced polypeptides into a racemic mixture. In certain embodiments, the combining is under conditions suitable for formation of the rippled antiparallel cross-β dimers of the present disclosure. For example, according to some embodiments, provided are methods that comprise combining (L,L,L)-(FX₁F)ₖ and (D,D,D)-(FX₂F)ₖ in a mixture under conditions in which rippled antiparallel cross-β dimers comprising (L,L,L)-(FX₁F)ₖ and (D,D,D)-(FX₂F)ₖ are formed. Examples of such (L,L,L)-(FX₁F)ₖ and (D,D,D)-(FX₂F)ₖ include those described above and in the Experimental section below. According to some embodiments, the combining is under conditions suitable for formation of a rippled β-sheet fibril of the present disclosure. Non-limiting examples of suitable conditions for forming the rippled antiparallel cross-β dimers and rippled β-sheet fibrils of the present disclosure are described in detail below.

Notwithstanding the appended claims, the present disclosure is also defined by the following embodiments:

1. A rippled antiparallel cross-β dimer comprising (L,L,L)-(FX₁F)ₖ dimerized with (D,D,D)-(FX₂F)ₖ, wherein X₁ and X₂ are independently selected from any amino acid, and wherein k is an integer of 1 or greater.
2. The rippled antiparallel cross-β dimer of embodiment 1, wherein X₁ and X₂ are independently selected from the group consisting of: F, Y, and W.
3. The rippled antiparallel cross-β dimer of embodiment 1 or embodiment 2, wherein X₁ and X₂ are the same.
4. The rippled antiparallel cross-β dimer of embodiment 1 or embodiment 2, wherein X₁ and X₂ are different.
5. The rippled antiparallel cross-β dimer of any one of embodiments 1 to 4, wherein the dimer is water-soluble.
6. The rippled antiparallel cross-β dimer of embodiment 5, wherein the N-terminus of the (L,L,L)-(FX₁F)ₖ monomer comprises a free amine or a free carboxylate.
7. The rippled antiparallel cross-β dimer of embodiment 5 or embodiment 6, wherein the N-terminus of the (D,D,D)-(FX₂F)ₖ monomer comprises a free amine or a free carboxylate.
8. A rippled β-sheet fibril comprising a plurality of the rippled antiparallel cross-β dimers of any one of embodiments 1 to 7.
9. A material comprising a plurality of the rippled antiparallel cross-β dimers of any one of embodiments 1 to 7.
10. A material comprising the rippled β-sheet fibril of embodiment 8.
11. The material of embodiment 9 or embodiment 10, wherein the material comprises the plurality of rippled antiparallel cross-β dimers held together by a combination of interdimer hydrogen bonds, ionic interactions, and van der Waals interactions.
12. A composition comprising the material of any one of embodiments 9 to 11.
13. The composition of embodiment 12, wherein the dimer or material is present in a liquid medium.
14. The composition of embodiment 13, wherein the liquid medium is an aqueous liquid medium.
15. A method comprising:
producing a polypeptide comprising, consisting essentially of, or consisting of, (L,L,L)-(FX₁)ₖ; and
producing a polypeptide comprising, consisting essentially of, or consisting of, (D,D,D)-(FX₂F)k,
wherein (L,L,L)-(FX₁F)ₖ and (D,D,D)-(FX₂F)ₖ are as defined in any one of embodiments 1 to 7.
   16. The method according to embodiment 15, wherein the polypeptides are produced by chemical synthesis.
   17. The method according to embodiment 16, wherein the chemical synthesis is by solid-phase polypeptide synthesis.
   18. The method according to embodiment 17, wherein the solid-phase polypeptide synthesis is Fmoc-based solid-phase peptide synthesis.
   19. The method according to any one of embodiments 15 to 18, further comprising purifying the produced (L,L,L)-(FX₁F)ₖ and (D,D,D)-(FX₂F)ₖ.
   20. The method according to any one of embodiments 15 to 19, further comprising combining the produced (L,L,L)-(FX₁F)ₖ and (D,D,D)-(FX₂F)ₖ into a racemic mixture.
   21. A method comprising:
combining (L,L,L)-(FX₁F)ₖ and (D,D,D)-(FX₂F)ₖ in a mixture under conditions in which rippled antiparallel cross-β dimers comprising (L,L,L)-(FX₁F)ₖ and (D,D,D)-(FX₂F)ₖ are formed,
wherein (L,L,L)-(FX₁F)ₖ and (D,D,D)-(FX₂F)ₖ are as defined in any one of embodiments 1 to 7.
   22. The method according to embodiment 21, wherein a rippled β-sheet fibril comprising the rippled antiparallel cross-β dimers is formed.

The following examples are offered by way of illustration and not by way of limitation.

### Experimental

### Example 1—Dimeric Antiparallel Rippled Sheets

The rippled sheet is a structural motif hypothesized by Pauling and Corey in 1953, in which extended peptidic β-strands associate with their mirror-images. For the rational design of rippled sheet nanomaterials to become tractable, crystal structures are needed. Reported herein is that a racemic mixture of (L,L,L)- and (D,D,D)-triphenylalanine, yields crystals that are built from periodically repeating rippled sheet dimers. Mining of the PDB reveals three further rippled sheet-containing crystal structures that had escaped the attention of the field thus far.

### Choice of System

The significance of the oligomeric phenylalanine motif for amyloid formation is well-established. For example, it is known that the hydrophobic LVFFA segment that spans the amino acid residues 17-21 of the Amyloid β (i.e., Aβ17-21) peptide is crucial for As fibrillization.³¹ Furthermore, Kiessling and coworkers have taken advantage of this by using the KLVFF segment for molecular recognition studies with Aβ.^{31,32} Reductionist studies of Aβ by Gazit and co-workers demonstrated that the short diphenylalanine peptide is itself capable of forming amyloid nanostructures.³³ Unlike the dipeptide, FF, which has been shown to form water-filled nanovesicles and hollow tubes, the tripeptide, FFF, spontaneously assembles into a diverse set of supramolecular assemblies depending on conditions, such as solid nanospheres, nanorods, helical-ribbons, plates, dendrimers, and doughnuts,³⁴⁻³⁶ similar to what has been reported for Aβ,³⁷ making it an interesting candidate from the standpoint of rippled sheet design. Additionally, Gazit and coworkers found that FFF demonstrated improved stability and peptide-network propensity over FF.³⁶ The authors also reported Thioflavin T (ThT) positivity for the FFF assemblies, indicative of ordered β-sheet content.³⁶

More recently, Nilsson and co-workers demonstrated that the Aβ16-22 segment, KLVFFAE, rapidly formed precipitates when mixed with its mirror-image counterpart klvffae, which the authors ascribed to rippled sheet formation based on isotope-edited FT-ICR mass spectrometric and solid state NMR spectroscopic experiments.²⁵

Peptides containing bulky, hydrophobic amino acids Phe (F), Val (V), Ile (I) and Leu (L) are believed to be particularly prone to forming rippled sheets.¹⁷ Phenylalanine stands out because of its relative rigidity, which should favor crystallization.³¹ A racemic mixture of (L,L,L)-triphenylalanine and (D,D,D)-triphenylalanine (i.e., FFF:fff) was chosen as the model. The N- and C-termini of FFF and fff were kept as free amines and free carboxylates, respectively, to afford peptides that (a) are water-soluble and (b) favor a defined antiparallel arrangement due to Coulombic attraction. Peptides were made on solid supports and purified using a procedure similar to one previously developed for Aβ purification.³⁹

### The FFF:Fff Dimer Structure

Combination of concentrated solutions of FFF and fff led to rapid formation of a fine precipitate. Optimization of conditions led to a protocol, in which controlled cooling of a solution saturated with a racemic mixture of FFF and fff from 75 to 25° C. at a rate of 0.1° C. min⁻¹ afforded single-crystal needles with length exceeding 3 mm. A short needle, suitable for single crystal X-ray diffraction was selected and the metric symmetry and Laue symmetry of the diffraction pattern obtained with Cu K_{α} radiation revealed that the crystal belonged to the monoclinic crystal system. Strict observance of Friedel's Law and the <E²−1>value of 1.008 indicate that the crystal is centrosymmetric, suggesting that the molecules had crystallized as the racemic compound. Centrosymmetry was confirmed by analysis of the systematic absences, which unambiguously confirmed the space group to be P2₁/c. The structure was solved using intrinsic phasing and refined against 0.84 Å-resolution data. The resolution and quality of the data permitted anisotropic refinement of all non-H atoms and semi-free refinement of H-atom positions.

The asymmetric unit contains a single tripeptide in its zwitterionic form. Both amides assume the expected trans configuration. The ψ angles of 114.6(2°) and 132.3(1)° and the φ angles of −124.7(4)° and −155.1(1)° for FFF fall within the range typically observed for β-pleated sheets. The side chains of the three residues assume, from N to C terminus, the gauche⁺(X₁=−63.2(2)°), trans (X₁=−175.1(1)°), and gauche (X₁=70.7(2)°) configurations.

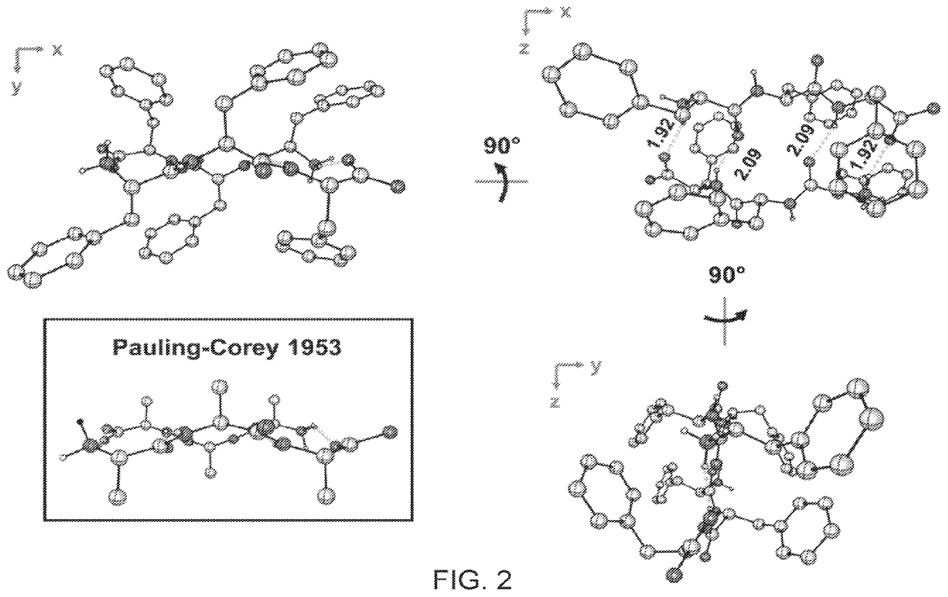

The dimer resides on a crystallographic inversion center, across which FFF and fff form two symmetry-related pairs of hydrogen bonds (FIG. 2). The terminal ammonium and carboxylate groups form a salt bridge with a N . . . O distance of 2.7660(18) Å and a N—H . . . O angle of 152.8(19)°. The hydrogen bond formed between the neutral amide units features an expectedly longer N . . . O distance of 2.9097(18) Å and a N—H . . . O angle of 157.4(17)°. The hydrogen bonds comprise the only significant intermolecular contacts between the components of the dimer; the torsion angles assumed by each of the phenylalanine units allow them to effectively interleave given the inversion symmetry relating the two molecules. This arrangement of hydrogen bonds is in excellent agreement with the model put forward by Pauling and Corey (FIG. 2). In that original work, they model the antiparallel rippled sheet using a translation of 7.00 Å, which agrees well with the C_{α,1} . . . C_{α,3} distance of 6.888(2) A in the present crystal structure.

### Crystal Lattice Analysis

The crystal is held together by a combination of interdimer hydrogen bonds, ionic interactions, and van der Waals interactions. In addition to interacting with the terminal carboxylate of the inversion-generated dimer mate, the terminal ammonium also forms hydrogen bonds to a glide-generated carbonyl of an enantiomeric tripeptide molecule (N . . . O=2.7244(17) A) and to the screw-generated terminal carboxylate of a molecule of identical handedness (N . . . O=2.6645(18) Å). The internal amide N—H unit that is not involved in the antiparallel cross-β FFF:fff dimer also hydrogen bonds to this same screw-generated terminal carboxylate (N . . . O=3.0168(17) Å). The H-atom positions in the final model are consistent with this hydrogen bonding pattern.

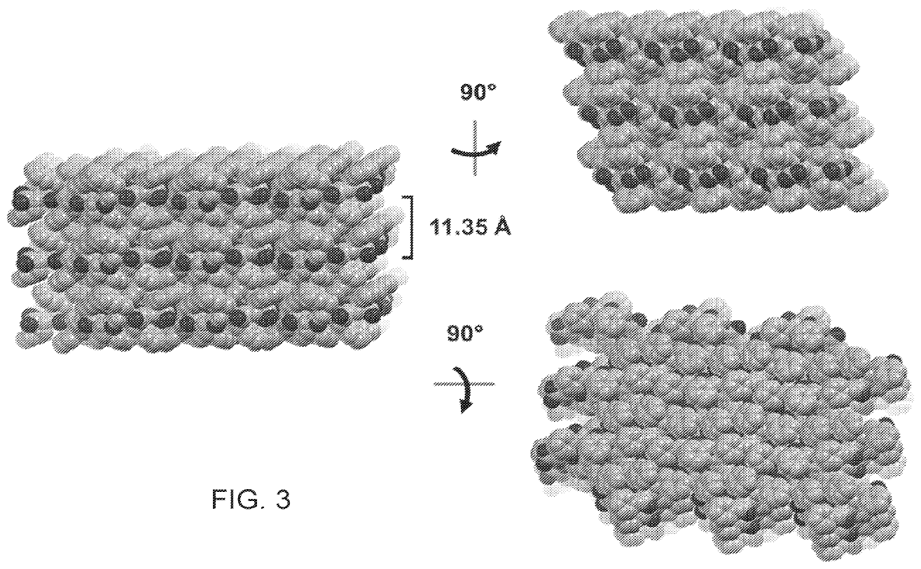

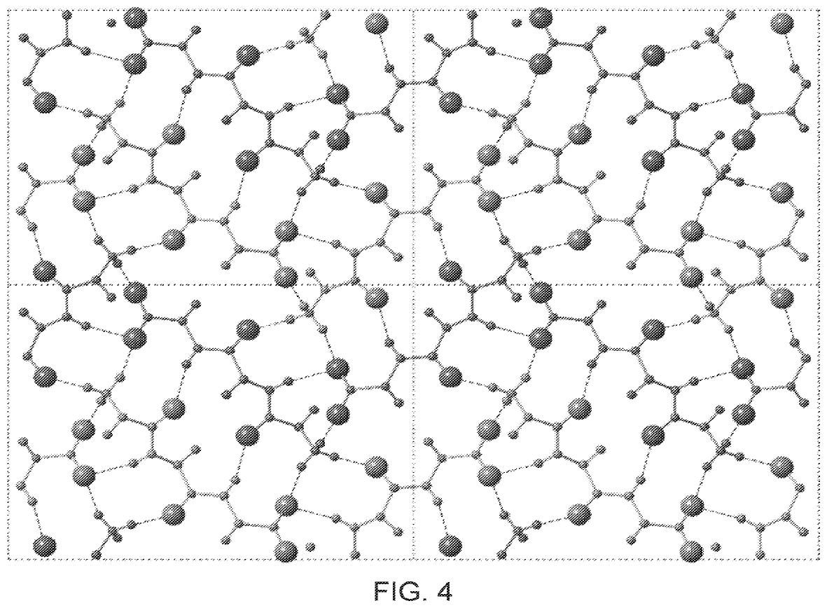

These hydrogen bonds extend to form sheets parallel to the crystallographic bc plane (FIG. 3). These sheets feature a hydrophilic core bounded on both sides by hydrophobic layers. The layers stack on one another with an interlayer spacing corresponding to the crystallographic a lattice parameter of 11.3563(5) Å. This nanoscale architecture, with clear alternation between hydrophobic and hydrophilic layers, is reminiscent of a phase separation. The dimeric rippled sheets were not found to assemble into extended “fibrillary” rippled sheets with long-range order, packing into a classic herringbone pattern instead (FIG. 4).

To confirm that an isolated FFF:fff rippled antiparallel cross-β dimer is in itself a stable arrangement, the dimer was subjected to full geometry optimization using Density Functional Theory (DFT) methods. The optimization produced only marginal local structural changes, confirming that the structural features of the dimer are inherent to the β-rippled-sheet hydrogen bonding pattern and not crystal packing forces. This result stands in good agreement with previous computational work on related rippled interfaces.^{8,17,40,41}

### Discussion

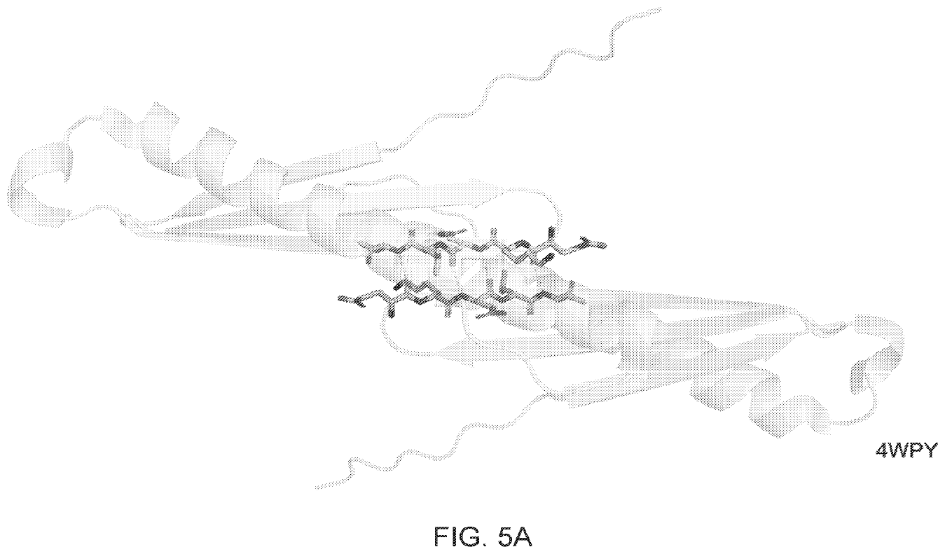

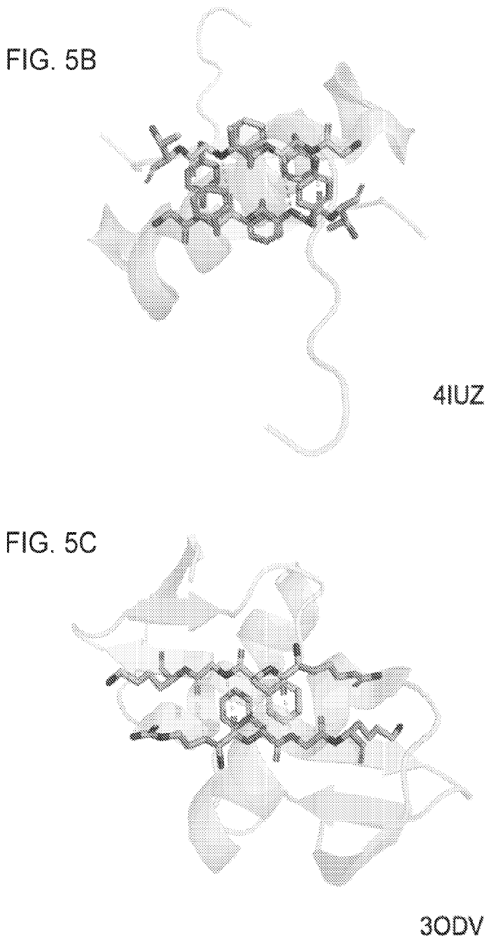

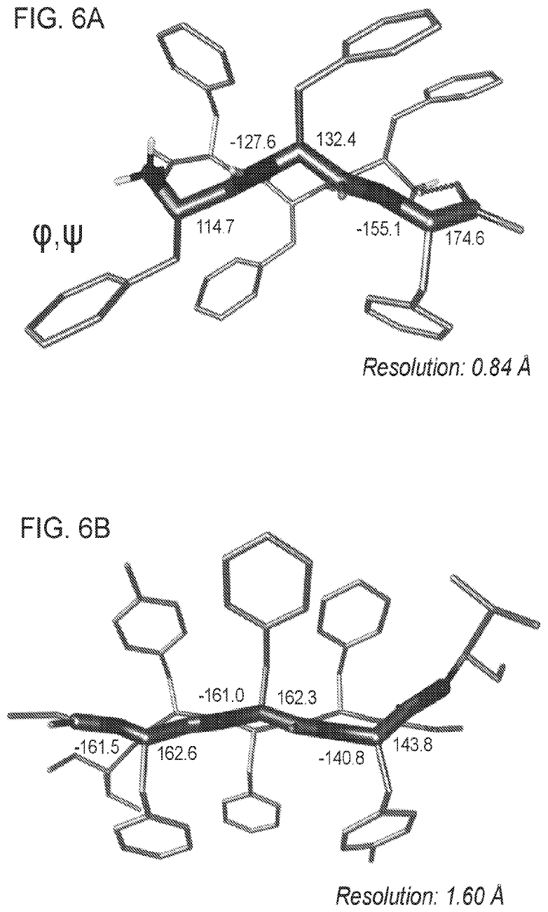

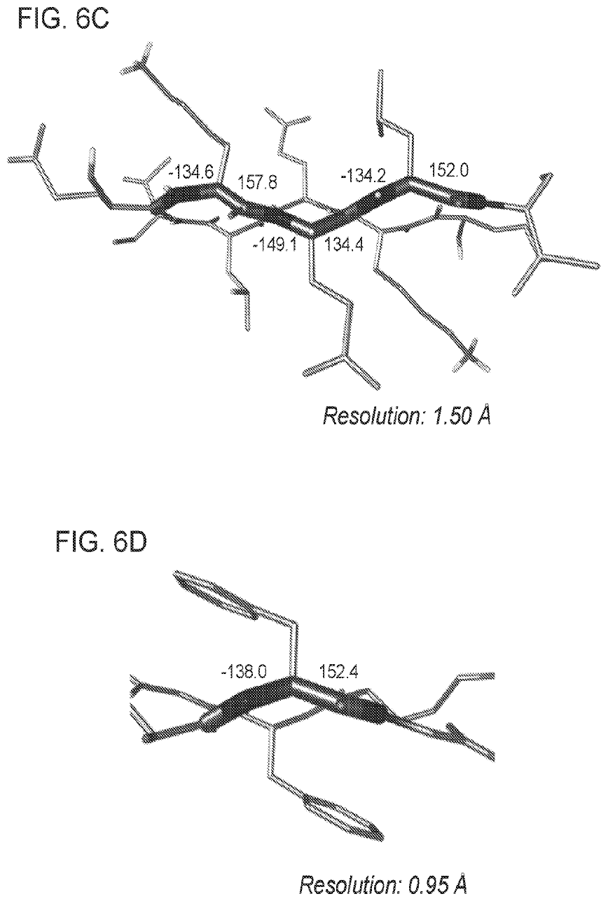

Presented above is a range of structural features gleaned from a crystal-structural analysis of the FFF:fff lattice. This is believed to be the first time that a rippled sheet crystal structure is being discussed in the literature. However, owing to the efforts of racemic protein crystallography, many crystal structures that contain potentially interacting mirror-image protein pairs are now available. It seemed plausible that the enantiomers in some of those structures might interact via rippled sheets. This possibility was interrogated by searching the Cambridge Structural Database (CSD) and the Protein Data Bank (PDB), as described in the Materials and Methods section. The CSD search revealed no rippled sheet structures. The PDB search identified three racemic protein crystal structures with a qualitative appearance suggesting the presence of antiparallel rippled sheets. Analysis of the three structures validated that dimeric rippled sheets were indeed present in all three cases (FIG. 5). As such, it was found that in the racemic crystal structure of the Rv1738 protein, the protein enantiomers interact through an antiparallel rippled sheet formed by the Lys-Glu-Leu triad and its enantiomer (FIG. 5A).⁴² It was also found that, in the racemic ester insulin crystal structure, the enantiomers are bridged by a rippled sheet formed between the Phe-Phe-Tyr triad and its enantiomer (FIG. 5B).⁴³ Finally, a very short rippled sheet segment of only one Phe residue and its enantiomer was observed in the racemic crystal structure of kaliotoxin (FIG. 5C).⁴⁴ Whereas in those three structural studies, the authors did recognize there were mirror-image interactions between their protein pairs, none of them identified those interactions as rippled sheets, which may be why those important structural insights appear to have escaped the attention of the rippled sheet community thus far. To gain deeper insights into the backbone conformations associated with the four rippled antiparallel sheet structures, their Ramachandran angles were analyzed (FIG. 6). It was noted that three of the rippled sheets contain internal L-Phe:D-Phe pairs, i.e., (F:::f). Their Ramachandran angles range from φ=−127.6° and ψ=132.4° with FFF:fff (FIG. 6A) to φ=−161.0° and ψ=162.3° with racemic ester insulin (FIG. 6B). This means that there is significant flexibility that is available to the (F:::f) pair in the context of the antiparallel rippled sheet, which may become a useful design element if the interest of the materials community to the rippled sheet motif continues to grow.

Pleated β-sheets are often observed in fibrils formed by aggregating enantiopure peptides, where they tend to display a one-dimensional long-range order. Numerous structures are available through the work of the Eisenberg lab on steric zippers and related systems.⁴⁵⁻⁵⁰ In contrast to the long-range packing noted in the Eisenberg systems, dimeric antiparallel rippled sheets were observed with FFF:fff (FIG. 2), but those dimers did not form extended rippled sheets (FIGS. 3 and 4). The lack of extended sheets may also be rooted in the hydrophobicity of the FFF:fff dimer that leads it to precipitate from water before it can mature into an extended fibrillary rippled sheet. Systematic optimization of crystallization parameters, including concentration, solvent identity, temperature, as well as variations in sequence, may allow the synthesis of extended fibrillary rippled sheet networks in the future. In that context it is interesting to compare the FFF:fff dimer structure with (a) the racemic Aβ40 structure, published in a recent collaborative study by the Raskatov and Tycko labs,²⁹ and (b) the hydrophobic Aβ16-22 segment in its interactions with its mirror-image, studied by the Nilsson lab. All three systems contain rippled antiparallel dimers, which is likely due, at least in part, to Coulombic attractions. However, there are important differences. Racemic Aβ40 forms fibrils with three Aβ40 units per layer and a fibril thickness of 7±1 nm.⁸ The crystalline Aβ16-22 aggregates, on the other hand, are micron-wide, which is consistent with the presence of thousands of peptides per layer.²⁵ Future X-ray structural studies of racemic Aβ16-22 should determine whether it (a) forms extended rippled sheets, (b) aggregates into rippled antiparallel cross-β dimers that then pack in ways similar to FFF:fff, or (c) packs in a way that is completely different.

The findings herein should be put in context with the recent paper by Liu and Gellman, where peptides designed to form two-stranded β-hairpins, composed of half L and half D residues did not exhibit any heterochiral stand pairing detectable by solution NMR.²⁴ It is noteworthy that one of the systems studied by the authors contained the VFF motif that is present in Aβ and is believed to be important for racemic Aβ fibrillization (i.e., Aβ Chiral Inactivation, Aβ-CI).^{7,25,29} The VFF motif is also very similar in terms of its size and hydrophobicity to the FFF motif studied here. A possible reason for the apparent discrepancy is that in Gellman's work, the L- and D-sequences were linked together, which may have induced a preference for homochiral strand pairing. Possibly more significantly, FFF:fff crystallization (similarly to Aβ-CI and the racemic Aβ16-22 model system studied by Nilsson) appears to occur under kinetic control, whereas the foldamers of the Gellman hairpin were monitored under thermodynamic equilibrium conditions. Similarly (albeit in the non-polar solvent CDCl₃), Chung and Nowick found that hydrophobic β-turn peptide mimics preferentially form homochiral (pleated) dimers.²³ Another important difference between the present work and the two solution NMR studies is that, in the present study, the rippled antiparallel FFF:fff dimers are packed into a three-dimensional crystal lattice that may, in itself, be a ripple-genic factor. In contrast, the solution NMR studies lacked evidence for the formation of higher order aggregates, and instead highlighted interactions between dimerizing peptide strands as isolated entities.

It may be tempting to ascribe the difference between the solution NMR experiments discussed above and the present findings to the fact that solution NMR work studied systems as pure dimers, whereas the present work produced extended layers, in which the individual dimers were stabilized through interactions with the crystal lattice. However, there is a crystal structure of the GSTSTA peptide in a racemic mixture with its enantiomer, in which self-sorting into pleated fibrillary structures was observed, showing that racemic aggregating peptide mixtures are not ripple-genic per se either.³⁰ In this specific case, it may have been because GSTSTA lacks bulky, hydrophobic groups that appear to promote rippled sheet formation.¹⁷ Yet it seems that the presence of bulky residues is not obligate either, as the first rippled sheet structure was reported for polyglycine I, which does not have sidechains.^{18,19} It should also be noted that, in addition to sequence, aggregation conditions are important. As such, it was noted with the MAX1:DMAX system developed by the Schneider lab, that the rigidity of the hydrogels formed depended on whether peptides were aggregated under kinetic or thermodynamic control, with thermodynamically controlled assembly producing the most rigid hydrogel systems.^{3,4,17} These are all conditions that should be explored in future research.

To summarize, presented herein are crystal-structural insights into a rippled sheet-based nanostructure obtained by temperature-controlled crystallization of FFF:fff. The structure consists of arrays of dimeric antiparallel rippled sheets, whose internal structural parameters agree well with the predictions by Pauling and Corey. The rippled dimers are arranged in a herringbone-pattern, into networks that are held together by in-plane salt bridges and hydrogen bonds and display lateral long-range segregation into hydrophobic and hydrophilic domains. Comparison of FFF:fff with the three orphaned rippled sheets identified by analyzing the racemic protein crystallography PDB supports the notion of Phe as a ripple-genic residue. Systematic exploration of Phe-containing racemic peptide mixtures may provide a rational framework on how to devise functional rippled sheet materials in the future.

### Materials and Methods for Example 1

Peptide Synthesis. The (L,L,L)-triphenylalanine (i.e., FFF) and (D,D,D)-triphenylalanine (i.e., fff) peptides were synthesized by standard Fmoc-based, solid-phase peptide chemistry, following previously reported protocols.^{39,51} Both peptides were synthesized using preloaded, Fmoc-phenylalanine 4-alkoxybenzyl alcohol Wang resin: Fmoc-L-Phe-Wang (Sigma) or Fmoc-D-Phe-Wang (Fisher). All syntheses were performed manually at 0.2 mM scale relative to resin loading. An orbital shaker was used for mixing in both the deprotection and coupling steps. The resin was swelled in 3 mL of dimethylformamide (DMF) in a filter tube, housing 250 mg Fmoc-Phe Wang resin (0.796 mmol/g loading) for 20 min. For Fmoc-deprotection, 30% piperidine (Spectrum) in DMF was added to the resin, and allowed to shake on an orbital shaker for 20 min. The deprotection solution was rinsed with DMF (3×) and dichloromethane (DCM, 2×) and the deprotection step was repeated. Coupling reagents used were 4 eq. N,N-diisopropylethylamine (Fisher), 3 eq. N,N,N′,N′-tetramethyl-O-(1H-benzotriazol-1-yl)uronium hexafluorophosphate (Fisher) and 3 eq. hydroxybenzotriazole hydrate (Oakwood Products). For amino acid coupling, 3 eq. of either Fmoc-L-Phe-OH (Fisher) or Fmoc-D-Phe-OH (ChemPep) with coupling reagents listed above were dissolved in 3 mL DMF and added to the reaction vessel, and allowed to shake for 30 min. The coupling step was repeated for each amino acid addition to improve yield. The aforementioned steps were repeated to produce the resin-bound tripeptides, NH₂-L-FFF-COOH and NH₂-D-fff-COOH. The peptides were cleaved and deprotected with a mixture consisting of trifluoroacetic acid (10 mL, Fisher), tri-isopropylsilane (1 mL, Fisher), and liquefied phenol (0.5 mL, Sigma). The peptide identities were confirmed with mass spectrometry. Peptides were purified by reverse-phase high-performance liquid chromatography (HPLC) with PLRP-S columns (Agilent), as previously described,^{39,51} yielding peptides with purities exceeding 95% (Figure S1-S2). HPLC was conducted under basic conditions (0.1% NH₄OH), to reduce aggregation and/or precipitation. Samples were lyophilized and stored as solid powders at −40° C.

Crystallization. Solutions of L-FFF and D-fff peptides were prepared separately by dissolving 7 mg of each individual peptide in 4 mL of nanopure water. The resulting solutions were sonicated and transferred to an oil bath at 90° C. and kept under stirring for one hour. To enhance dissolution of the cloudy slurries, 80 μL of hexafluoroisopropanol (HFIP; Fisher) was added to the solutions (2% of total volume), but significant cloudiness was still observed. After an additional 1 h of heating in the oil bath, the two individual peptide solutions were combined by adding D-fff to the L-FFF solution, dropwise. The resulting cloudy solution was rapidly transferred to a Teflon lined stainless steel autoclave, which was sealed and placed on an oven at 75° C. for 10 d followed by a slow colling process at a rate of 0.1° C./min, leading to the formation of colorless, needle-like crystals.

Single-Crystal X-ray Diffraction. A suitable colorless needle with dimensions of 0.1×0.09×0.03 mm³ was used for single-crystal X-ray diffraction data collection at 100 K on a Rigaku XtaLAB Synergy-S diffractometer using Cu K_{α} radiation (λ=1.54 Å). Data collection, processing and reduction were performed with CrysAlis^{Pro}.⁵² After face indexing, numerical absorption correction was applied using gaussian integration. Empirical absorption correction using spherical harmonics was applied using SCALE3 ABSPACK scaling algorithm. The structure was solved by intrinsic phasing using SheIXT and refined with SheIXL via Olex2.⁵³⁻⁵⁵ All non-hydrogen atoms were refined anisotropically using standard procedures.⁵⁶ Atomic displacement parameters for hydrogen atoms in the terminal amine group were fixed to 1.5(Uᵢₛₒ) of the attached nitrogen atom. For all other hydrogen atoms, the values were fixed to 1.2(Uᵢₛₒ) of the atoms to which they are attached. The N—H distances in the amine and amide groups were restrained to 0.91(2) Å and 0.88(2) Å, respectively. All other hydrogen atoms were placed at geometrically calculated positions and refined using a riding model.

Computational Chemistry. The input geometry for the optimization of FFF:fff was generated using the crystallographic data. The optimization was performed using ORCA 4.2.1, using Becke's 1988 exchange functional and Perdew's 1986 correlation functional (i.e., BP86)^{57,58} and the resolution of the identity approximation. Ahlrichs' def2-SVP basis set and the def2/J auxiliary basis set were used.^{59,60} An atom-pairwise dispersion correction with the Becke-Johnson damping scheme was applied (D3BJ).^{61,62} Implicit aqueous solvation was achieved using a conductor-like polarizable continuum model (CPCM=water).⁶³

CSD Search. A systematic search of the CSD (version 5.41) was performed using ConQuest (version 2.0.4). Two queries were submitted simultaneously. The first searched for a C(C)C(O)NHC(C)C(O)NHC(C)C(O)NH fragment with all bond types set to “any”, with both φ torsion angles from −180-0°, and with both ψ torsion angles within the range 0-180°. The second query required the presence of a distinct C(C)C(O)NHC(C)C(O)NHC(C)C(O)NH fragment with all bond types set to “any”, with both φ torsion angles from 0-180°, and with both ψ torsion angles within the range −180-0°. The hits from this search were inspected manually and none featured a rippled sheet motif.

PDB Structural Database Mining. The PDB database was searched for the term “Racemic”, and the results were narrowed by selecting “protein” as the polymer entity type, producing a total of 387 hits. The majority of those hits were, however, not truly racemic protein structures, but rather, enantiomerically pure proteins complexed with racemic molecules or simply included racemic compounds used during synthesis. These were excluded from the search. From the remaining hits, those in which the mirror-image proteins had β-strands oriented in ways that made them potentially capable of forming rippled sheets were manually selected.

This eventually produced three structures that can be accessed through the PDB via reference codes 4WPY⁴², 4IUZ⁴³, and 3ODV.⁴⁴

### REFERENCES FOR INTRODUCTION AND EXAMPLE 1

1 T. N. M. Schumacher, L. M. Mayr, D. L. Minor Jr., M. A. Milhollen, M. W. Burgess and P. S. Kim, Identification of D-Peptide Ligands Through Mirror-Image Phage Display, Science, 1996, 271, 1854-1857.
2 D. M. Eckert, V. N. Malashkevich, L. H. Hong, P. A. Carr and P. S. Kim, Inhibiting HIV-1 entry: Discovery of D-peptide inhibitors that target the gp41 coiled-coil pocket, Cell, 1999, 99, 103-115.
3 K. Nagy-Smith, P. J. Beltramo, E. Moore, R. Tycko, E. M. Furst and J. P. Schneider, Molecular, Local, and Network-Level Basis for the Enhanced Stiffness of Hydrogel Networks Formed from Coassembled Racemic Peptides: Predictions from Pauling and Corey, ACS Cent. Sci., 2017, 3, 586-597.
4 K. J. Nagy, M. C. Giano, A. Jin, D. J. Pochan and J. P. Schneider, Enhanced mechanical rigidity of hydrogels formed from enantiomeric peptide assemblies, J. Am. Chem. Soc., 2011, 133, 14975-14977.
5 D. Willbold and J. Kutzsche, Do we need anti-prion compounds to treat Alzheimer's disease?, Molecules, 2019, 24, 2237.
6 T. Van Groen, S. Schemmert, O. Brener, L. Gremer, T. Ziehm, M. Tusche, L. Nagel-Steger, I. Kadish, E. Schartmann, A. Elfgen, D. Jürgens, A. Willuweit, J. Kutzsche and D. Willbold, The Aβ oligomer eliminating D-enantiomeric peptide RD2 improves cognition without changing plaque pathology, Sci. Rep., 2017, 7, 16275.
7 S. Dutta, A. R. Foley, C. J. A. Warner, X. Zhang, M. Rolandi, B. Abrams and J. A. Raskatov, Suppression of Oligomer Formation and Formation of Non-Toxic Fibrils upon Addition of Mirror-Image Aβ42 to the Natural L-Enantiomer, Angew. Chemie.—Int. Ed., 2017, 56, 11506-11510.
8 S. Dutta, A. R. Foley, A. J. Kuhn, B. Abrams, H. W. Lee and J. A. Raskatov, New insights into differential aggregation of enantiomerically pure and racemic Aβ40 systems, Pept. Sci., 2019, 111, e24139.
9 A. R. Foley, G. P. Roseman, K. Chan, A. Smart, T. S. Finn, K. Yang, R. Scott Lokey, G. L. Millhauser and J. A. Raskatov, Evidence for aggregation-independent, PrPC-mediated Aβ cellular internalization, Proc. Natl. Acad. Sci. U.S.A., 2020, 117, 28625-28631.
10 A. R. Foley and J. A. Raskatov, Understanding and controlling amyloid aggregation with chirality, Curr. Opin. Chem. Biol., 2021, 64, 1-9.
11 T. O. Yeates and S. B. H. Kent, Racemic protein crystallography, Annu. Rev. Biophys., 2012, 41, 41-61.
12 K. W. Kurgan, A. F. Kleman, C. A. Bingman, D. F. Kreitler, B. Weisblum, K. T. Forest and S. H. Gellman, Retention of Native Quaternary Structure in Racemic Melittin Crystals, J. Am. Chem. Soc., 2019, 141, 7704-7708.
13 L. E. Zawadzke and J. M. Berg, The structure of a centrosymmetric protein crystal, Proteins Struct. Funct. Bioinforma., 1993, 16, 301-305.
14 B. L. Pentelute, Z. P. Gates, V. Tereshko, J. L. Dashnau, J. M. Vanderkooi, A. A. Kossiakoff and S. B. H. Kent, X-ray structure of snow flea antifreeze protein determined by racemic crystallization of synthetic protein enantiomers, J. Am. Chem. Soc., 2008, 130, 9695-9701.
15 L. Pauling and R. B. Corey, The pleated sheet, a new layer configuration of polypeptide chains, Proc. Natl. Acad. Sci. U.S.A., 1951, 37, 251-256.
16 L. Pauling and R. B. Corey, Two Rippled-Sheet Confirgurations of Polypeptide Chains, and a Note about the Pleated Sheets, Proc. Nat. Acad. Sci. U.S.A., 1953, 39, 253-256.
17 J. A. Raskatov, J. P. Schneider and B. L. Nilsson, Defining the Landscape of the Pauling-Corey Rippled Sheet: An Orphaned Motif Finding New Homes, Acc. Chem. Res., 2021, 54, 2488-2501.
18 B. Lotz, Crystal structure of polyglycine I, J. Mol. Biol., 1974, 87, 169-180.
19 F. Colonna-Cesari, S. Premilat and B. Lotz, Structure of polyglycine I: A comparison of the antiparallel pleated and antiparallel rippled sheets, J. Mol. Biol., 1974, 87, 181-191.
20 W. H. Moore and S. Krimm, Vibrational analysis of peptides, polypeptides, and proteins. I. Polygylcine I, Biopolymers, 1976, 15, 2439-2464.
21 I. Weissbuch, R. A. Illos, G. Bolbach and M. Lahav, Racemic β-sheets as templates of relevance to the origin of homochirality of peptides: Lessons from crystal chemistry, Acc. Chem. Res., 2009, 42, 1128-1140.
22 I. Rubinstein, R. Eliash, G. Bolbach, I. Weissbuch and M. Lahav, Racemic β sheets in biochirogenesis, Angew. Chemie.—Int. Ed., 2007, 46, 3710-3713.
23 D. M. Chung and J. S. Nowick, Enantioselective Molecular Recognition between β-Sheets, J. Am. Chem. Soc., 2004, 126, 3062-3063.
24 X. Liu and S. H. Gellman, Comparisons of β-Hairpin Propensity Among Peptides with Homochiral or Heterochiral Strands, ChemBioChem, 2021, 22, 2772-2776.
25 J. M. Urban, J. Ho, G. Piester, R. Fu and B. L. Nilsson, Rippled β-sheet formation by an amyloid-β fragment indicates expanded scope of sequence space for enantiomeric β-sheet peptide coassembly, Molecules, 2019, 24, 1983.
26 R. J. Swanekamp, J. T. M. Dimaio, C. J. Bowerman and B. L. Nilsson, Coassembly of enantiomeric amphipathic peptides into amyloid-inspired rippled β-sheet fibrils, J. Am. Chem. Soc., 2012, 134, 5556-5559.
27 A. M. Garcia, C. Giorgiutti, Y. El Khoury, V. Bauer, C. Spiegelhalter, E. Leize-Wagner, P. Hellwig, N. Potier and V. Torbeev, Aggregation and Amyloidogenicity of the Nuclear Coactivator Binding Domain of CREB-Binding Protein, Chem.—A. Eur. J., 2020, 26, 9889-9899.
28 V. Torbeev, M. Grogg, J. Ruiz, R. Boehringer, A. Schirer, P. Hellwig, G. Jeschke and D. Hilvert, Chiral recognition in amyloid fiber growth, J. Pept. Sci., 2016, 22, 290-304.
29 J. A. Raskatov, A. R. Foley, J. M. Louis, W.-M. Yau and R. Tycko, Constraints on the Structure of Fibrils Formed by a Racemic Mixture of Amyloid-β Peptides from Solid-State NMR, Electron Microscopy, and Theory, J. Am. Chem. Soc., 2021, 143, 13299-13313.
30 C.-T. Zee, C. Glynn, M. Gallagher-Jones, J. Miao, C. G. Santiago, D. Cascio, T. Gonen, M. R. Sawaya and J. A. Rodriguez, Homochiral and racemic MicroED structures of a peptide repeat from the ice-nucleation protein InaZ, IUCrJ, 2019, 6, 197-205.
31 J. Ghanta, C. L. Shen, L. L. Kiessling and R. M. Murphy, A strategy for designing inhibitors of β-amyloid toxicity, J Biol Chem, 1996, 271, 29525-29528.
32 T. L. Lowe, A. Strzelec, L. L. Kiessling and R. M. Murphy, Structure—Function relationships for inhibitors of β-Amyloid toxicity containing the recognition sequence KLVFF, Biochemistry, 2001, 40, 7882-7889.
33 S. Brahmachari, Z. A. Arnon, A. Frydman-Marom, E. Gazit and L. Adler-Abramovich, Diphenylalanine as a Reductionist Model for the Mechanistic Characterization of β-Amyloid Modulators, ACS Nano., 2017, 11, 5960-5969.
34 C. Guo, Y. Luo, R. Zhou and G. Wei, Triphenylalanine peptides self-assemble into nanospheres and nanorods that are different from the nanovesicles and nanotubes formed by diphenylalanine peptides, Nanoscale, 2014, 6, 2800-2811.
35 E. Mayans, J. Casanovas, A. M. Gil, A. I. Jiménez, C. Cativiela, J. Puiggalí and C. Alemán, Diversity and Hierarchy in Supramolecular Assemblies of Triphenylalanine: From Laminated Helical Ribbons to Toroids, Langmuir, 2017, 33, 4036-4048.
36 P. Tamamis, L. Adler-Abramovich, M. Reches, K. Marshall, P. Sikorski, L. Serpell, E. Gazit and G. Archontis, Self-assembly of phenylalanine oligopeptides: Insights from experiments and simulations, Biophys. J., 2009, 96, 5020-5029.
37 R. Roychaudhuri, M. Yang, M. M. Hoshi and D. B. Teplow, Amyloid-β protein assembly and Alzheimer disease, J. Biol. Chem., 2009, 284, 4749-4753.
38 J. Jaques, A. A. Collet and S. H. Wilen, Enantiomers, Racemates, and Resolutions, John Wiley & Sons, Ltd, 1991.
39 C. J. A. Warner, S. Dutta, A. R. Foley and J. A. Raskatov, A Tailored HPLC Purification Protocol That Yields High-purity Amyloid Beta 42 and Amyloid Beta 40 Peptides, Capable of Oligomer Formation, JoVE, 2017, e55482.
40 J. A. Raskatov, Conformational Selection as the Driving Force of Amyloid β Chiral Inactivation, ChemBioChem, 2020, 21, 2945-2949.
41 J. A. Raskatov, A DFT study of structure and stability of pleated and rippled cross-β sheets with hydrophobic sidechains, Biopolymers, 2021, 112, e23391.
42 R. D. Bunker, K. Mandal, G. Bashiri, J. J. Chaston, B. L. Pentelute, J. S. Lott, S. B. H. Kent and E. N. Baker, A functional role of Rv1738 in Mycobacterium tuberculosis persistence suggested by racemic protein crystallography, Proc. Natl. Acad. Sci. U.S.A., 2015, 112, 4310-4315.
43 M. Avital-Shmilovici, K. Mandal, Z. P. Gates, N. B. Phillips, M. A. Weiss and S. B. H. Kent, Fully convergent chemical synthesis of ester insulin: Determination of the high resolution X-ray structure by racemic protein crystallography, J. Am. Chem. Soc., 2013, 135, 3173-3185.
44 B. L. Pentelute, K. Mandal, Z. P. Gates, M. R. Sawaya, T. O. Yeates and S. B. H. Kent, Total chemical synthesis and X-ray structure of kaliotoxin by racemic protein crystallography, Chem. Commun., 2010, 46, 8174-8176.
45 M. R. Sawaya, S. Sambashivan, R. Nelson, M. I. Ivanova, S. A. Sievers, M. I. Apostol, M. J. Thompson, M. Balbirnie, J. J. W. Wiltzius, H. T. Mcfarlane, A. Ø. Madsen, C. Riekel and D. Eisenberg, Atomic structures of amyloid cross-β spines reveal varied steric zippers, Nature, 2007, 447, 453-457.
46 M. P. Hughes, M. R. Sawaya, D. R. Boyer, L. Goldschmidt, J. A. Rodriguez, D. Cascio, L. Chong, T. Gonen and D. S. Eisenberg, Atomic structures of low-complexity protein segments reveal kinked p sheets that assemble networks, Science, 2018, 359, 698-701.
47 E. L. Guenther, Q. Cao, H. Trinh, J. Lu, M. R. Sawaya, D. Cascio, D. R. Boyer, J. A. Rodriguez, M. P. Hughes and D. S. Eisenberg, Atomic structures of TDP-43 LCD segments and insights into reversible or pathogenic aggregation, Nat. Struct. Mol. Biol., 2018, 25, 463-471.
48 D. Li, E. M. Jones, M. R. Sawaya, H. Furukawa, F. Luo, M. Ivanova, S. A. Sievers, W. Wang, O. M. Yaghi, C. Liu and D. S. Eisenberg, Structure-based design of functional amyloid materials, J. Am. Chem. Soc., 2014, 136, 18044-18051.
49 J. A. Rodriguez, M. I. Ivanova, M. R. Sawaya, D. Cascio, F. E. Reyes, D. Shi, S. Sangwan, E. L. Guenther, L. M. Johnson, M. Zhang, L. Jiang, M. A. Arbing, B. L. Nannenga, J. Hattne, J. Whitelegge, A. S. Brewster, M. Messerschmidt, S. Boutet, N. K. Sauter, T. Gonen and D. S. Eisenberg, Structure of the toxic core of α-synuclein from invisible crystals., Nature, 2015, 525, 486-490.
50 L. Saelices, L. M. Johnson, W. Y. Liang, M. R. Sawaya, D. Cascio, P. Ruchala, J. Whitelegge, L. Jiang, R. Riek and D. S. Eisenberg, Uncovering the mechanism of aggregation of human transthyretin, J. Biol. Chem., 2015, 290, 28932-28943.
51 A. J. Kuhn, B. S. Abrams, S. Knowlton and J. A. Raskatov, Alzheimer's Disease ‘non-amyloidogenic’ p3 Peptide Revisited: A Case for Amyloid-α, ACS Chem. Neurosci., 2020, 11, 1539-1544.
52 Rigaku, 2020, CrysAlisPro 1.171.41.110a.
53 G. M. Sheldrick, SHELXT—Integrated space-group and crystal-structure determination, Acta Crystallogr. Sect. A Found. Crystallogr., 2015, 71, 3-8.
54 G. M. Sheldrick, Crystal structure refinement with SHELXL, Acta Crystallogr. Sect. C, 2015, 71, 3-8.
55 O. V. Dolomanov, L. J. Bourhis, R. J. Gildea, J. A. K. Howard and H. Puschmann, OLEX2: A complete structure solution, refinement and analysis program, J. Appl. Crystallogr., 2009, 42, 339-341.
56 P. Muller, Practical suggestions for better crystal structures, Crystallogr. Rev., 2009, 15, 57-83.
57 A. D. Becke, Density-functional exchange-energy approximation with correct asymptotic behavior, Phys. Rev. A, 1988, 38, 3098-3100.
58 J. P. Perdew, Density-functional approximation for the correlation energy of the inhomogeneous electron gas, Phys. Rev. B, 1986, 33, 8822-8824.
59 F. Weigend and R. Ahlrichs, Balanced basis sets of split valence, triple zeta valence and quadruple zeta valence quality for H to Rn: Design and assessment of accuracy, Phys. Chem. Chem. Phys., 2005, 7, 3297-3305.
60 F. Weigend, Accurate Coulomb-fitting basis sets for H to Rn, Phys. Chem. Chem. Phys., 2006, 8, 1057-1065.
61 S. Grimme, J. Antony, S. Ehrlich and H. Krieg, A consistent and accurate ab initio parametrization of density functional dispersion correction (DFT-D) for the 94 elements H—Pu, J. Chem. Phys., 2010, 132, 1-19.
62 S. Grimme, S. Ehrlich and L. Goerigk, Effect of the Damping Function in Dispersion Corrected Density Functional Theory, J. Comput. Chem., 2011, 32, 1456-1465.
63 M. Garcia-Ratés and F. Neese, Efficient implementation of the analytical second derivatives of hartree-fock and hybrid DFT energies within the framework of the conductor-like polarizable continuum model, J. Comput. Chem., 2019, 40, 1816-1828.

### Example 2—the Rippled β-Sheet Layer Configuration: A Novel Supramolecular Architecture

Tailored proteins and peptides hold immense potential for materials development and biomedical application. Chiral (i.e., D-amino acid) substitutions may be employed to generate self-assembling peptide architectures with unique properties,¹⁻⁶ bioactive compounds with distinct activities,⁷⁻¹³ as well as systems with enhanced crystallization behavior.¹⁴⁻¹⁸ There is great interest in developing new peptidic systems that contain D-amino acid substitutions, as this would allow systematic access to a vast structural space with unique molecular properties.

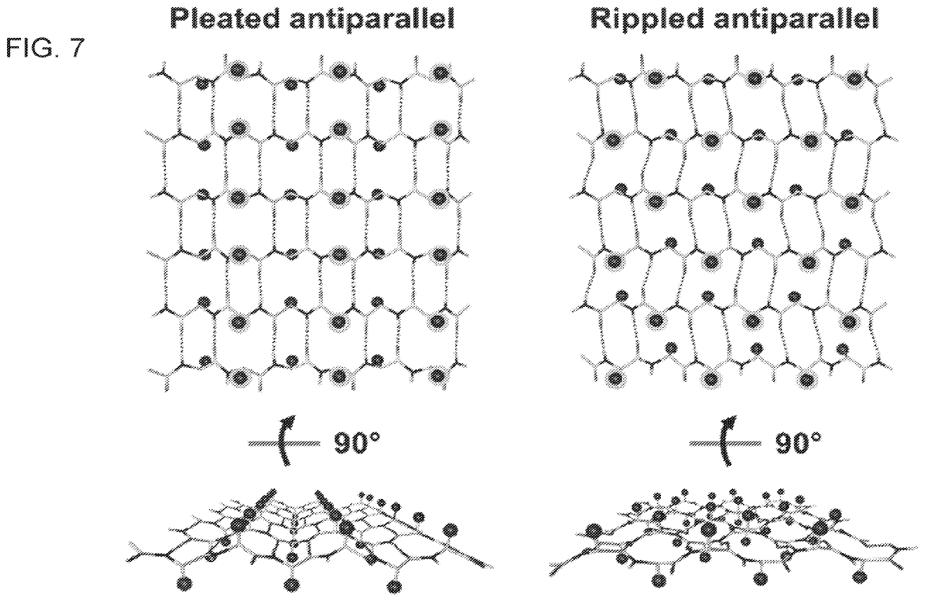

The rippled β-sheet is a largely neglected structural motif, hypothesized by Pauling and Corey in 1953.¹⁹ It is closely related to but distinct from the pleated β-sheet proposed by the same authors two years prior.²⁰ More specifically, the pleated β-sheet is homochiral., i.e., enantiopure, whereas in the rippled β-sheet, every second peptide strand is of opposing chirality, i.e., racemic (FIG. 7).

Research conducted in the 1970s combined experiment and theory, finding that the achiral polyglycine peptide forms chiral conformers and associates into rippled β-sheets known as polyglycine I.²¹⁻²³ This work was reviewed recently by Lotz, who performed much of this early pioneering research.²⁴ The ensuing four decades were characterized by only minimal research activity in the field.²⁵⁻²⁷ From 2010 on, the laboratories of Schneider, Nilsson and Raskatov independently published a number of key studies.^{1-5, 11, 28-31} Much of this work was reviewed in an article written jointly by the three investigators.³²

U.S. Patent Application No. 63/211,980 described the first X-ray crystal structural study of a rippled β-sheet, reporting that (L,L,L)-triphenylalanine (i.e., “FFF”) and (D,D,D)-triphenylalanine (i.e., fff) form a rippled β-sheet dimer (i.e., “FFF:fff”).³³ However, instead of forming extended rippled β-sheet layers (i.e., rippled β-sheet fibrils) predicted by Pauling and Corey, the FFF:fff dimers packed into a herringbone structure, thus raising doubt whether the rippled β-sheet fibril was a viable supramolecular architecture. The present example reports on three crystal structures of periodic rippled β-sheets, formed from three distinct peptide systems. These findings answer a longstanding structural question and pave the way to systematic structure-based design of a new class of supramolecular polymers.

Although the present understanding of what makes a peptide ripple-genic remains very limited, some design principles are beginning to emerge. As such, the X-ray crystallography work described in Example 1 above identified phenylalanine as a ripple-genic residue.³³ Although the FFF:fff model successfully formed cross-β dimers, the system stopped short of forming periodic β-sheet layers, possibly due to a quirk in the packing of the phenylalanine side chains, favoring end-to-edge rather than edge-to-edge interactions. To explore a greater sequence space, tripeptides with different aromatic amino acids were designed.

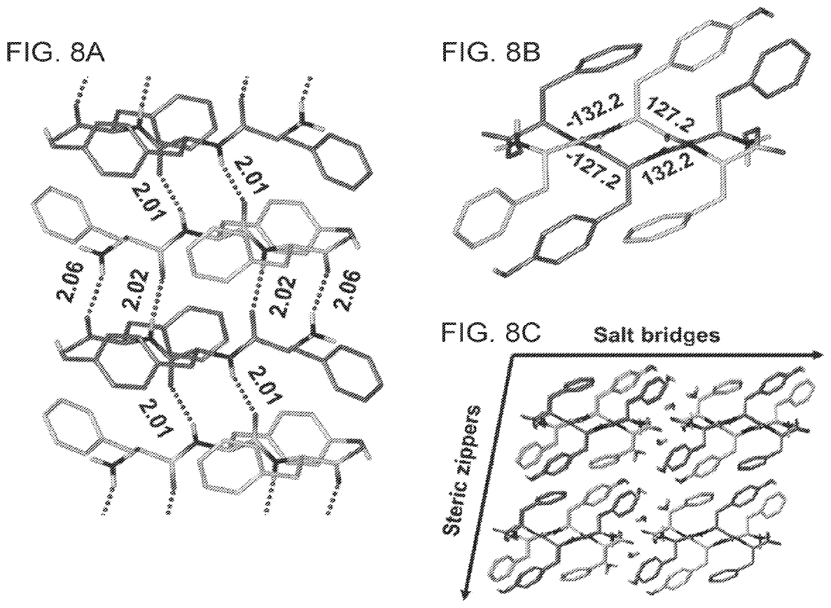

The desired outcome was achieved by substituting the central phenylalanine residues within FFF and fff by tyrosine (FYF and fyf, respectively). The racemic mixture of FYF and its mirror-image counterpart, fyf, crystallized into small needles from a water/hexafluoroisopropanol (HFIP) mixture, which were revealed by X-ray crystallography to be composed of periodic antiparallel rippled β-sheet layers (FIG. 8). The asymmetric unit of the FYF:fyf crystal is a single tripeptide. The individual tripeptides stack in the H-bonding dimension, forming extended antiparallel rippled β-sheet layers, in which mirror-image peptide strands are arranged in strictly alternating fashion. This novel layer architecture, which is termed herein as [FYF:fyf]n, is in excellent qualitative agreement with the prediction made by Pauling and Corey. Each L-tripeptide is sandwiched between two D-tripeptides, and each D-tripeptide is sandwiched between two L-tripeptides in periodic fashion within the individual β-sheet layers, with H-bond distances ranging from 2.01 Å to 2.06 Å (FIG. 8A). Each tripeptide has four H-bonds to one of its two direct neighbors in the layer (i.e., “tight dimer”), and two H-bonds to the other (i.e., “loose dimer”). The tight FYF:fyf rippled β-sheet dimer closely resembles the FFF:fff rippled β-sheet dimer.³³. The resemblance suggests that the initial step of mirror-image peptide self-assembly may be the formation of the tight dimer, which then nucleates sequence-dependent higher order assembly. The torsional angles for the central residue are measured as φ=−132.2° and ψ=127.2° for the L-tripeptide, and φ=132.2° and ψ=−127.2° for the inversion-related D-tripeptide (FIG. 8B). The lack of twist is in agreement with theory.²⁹ The individual rippled antiparallel [FYF:fyf]n β-sheet columns associate periodically to form a three-dimensional crystallographic lattice (FIG. 8C). Two distinct lateral association modes are observed, one of which is governed by salt bridges between the two peptide enantiomers, and the other which is driven by hydrophobic steric zippers between the mirror-image peptides. Given the analogy to amyloids,³⁴ the pairs of rippled β-sheets that are mated via steric zippers and extend along the length of the needle crystal are defined as rippled β-sheet fibrils.

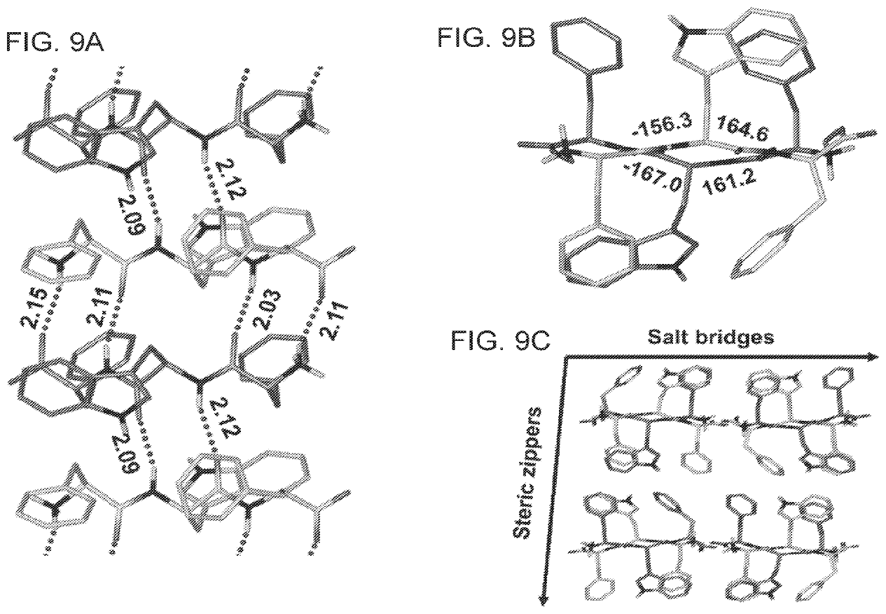

To determine whether rippled β-sheet fibrils would tolerate a bulkier sidechain, the central residue was changed to Trp (“W”). The racemic mixture of FWF and fwf was crystallized, yielding needles containing periodic [FWF:fwf]n antiparallel rippled β-sheet layers (FIG. 9). H-bond distances between peptide strands within the [FWF:fwf]n columns are slightly longer than with [FYF:fyf]n and cover a somewhat wider range from 2.03 Å to 2.15 Å (FIG. 9A). Once again, an alternation between tight and loose dimers is noted. Unlike the [FYF:fyf]n structure, the asymmetric unit of [FWF:fwf]n contains two crystallographically independent enantiomeric peptides that deviate slightly from perfect inversion symmetry. The torsional angles for the central residue are measured as φ=−156.3° and ψ=164.6° for the L-tripeptide, and φ=161.2° and ψ=−167.0° for the D-tripeptide (FIG. 9B; note that for each fibril there is a mirror-image fibril in the lattice, cf. FIG. 9C). The packing of individual rippled β-sheet layers in the [FWF:fwf]n lattice is similar to that of [FYF:fyf]n (FIG. 9C vs FIG. 8C), with pairs of [FWF:fwf]n layers mated via steric zippers to form rippled β-sheet fibrils. Similarly to [FYF:fyf]n, the rippled β-sheet fibrils of [FWF:fwf]n are associated laterally through salt bridges.

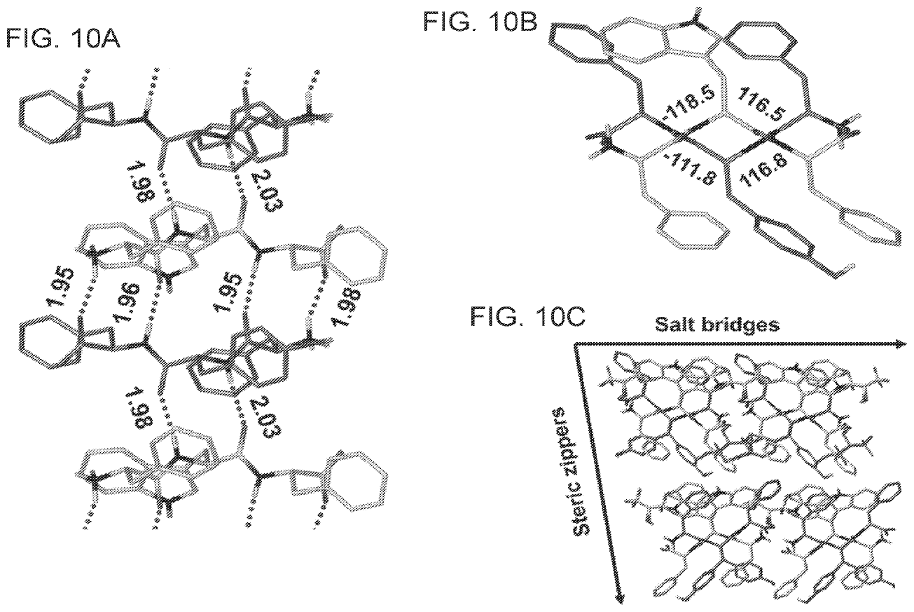

These findings prompted the exploration of the possibility of making a quasi-racemic rippled β-sheet fibril as proof of concept for an [A:B]n array, a useful new design principle for peptidic materials.⁶ An equimolar mixture of the FWF L-tripeptide and the fyf D-tripeptide was crystallized, and yielded needles containing periodic [FWF:fyf]n rippled antiparallel β-sheet layers, in which the L- and the D-tripeptides were found to strictly alternate (FIG. 10). The backbone configuration is very similar to that found with [FYF:fyf]n and [FWF:fwf]n. The H-bond distances within the rippled β-sheet layer range from 1.95 Å to 2.03 Å. An alternation of tight and loose interfaces is once again noted, with four and two H-bonds, respectively (FIG. 10A). The torsional angles for the central residue are measured as φ=−118.5° and ψ=116.5° for the FWF L-tripeptide, and φ=116.8° and ψ=−111.8° for the fyf D-tripeptide (FIG. 10B). Like the systems discussed above, the individual [FWF:fyf]n columns are mated via steric zipper interfaces into rippled β-sheet fibrils that are packed laterally through salt bridges (FIG. 10C). Two L-Tryptophan rotamers and two D-Tyrosine rotamers are present (FIG. 10C; only one set of rotamers is shown in FIG. 10A; other rotamer set no shown). Comparison of the Ramachandran (phi/psi) angles associated with the central residues was also of interest, revealing that the individual [FWF:fwf]n rippled β-sheet layers are substantially flatter than both [FYF:fyf]n and [FWF:fyf]n (cf. FIGS. 8B, 9B and 10B). A possible explanation is that in the case of [FWF:fwf]n, the structure adopts an extended conformation to enable aromatic stacking of F1 and w2 side chains on neighboring strands. The other two rippled β-sheet systems reveal no such stacking of aromatic rings. Substantial phi/psi angle variance has been previously noted in pleated β-sheets.³¹

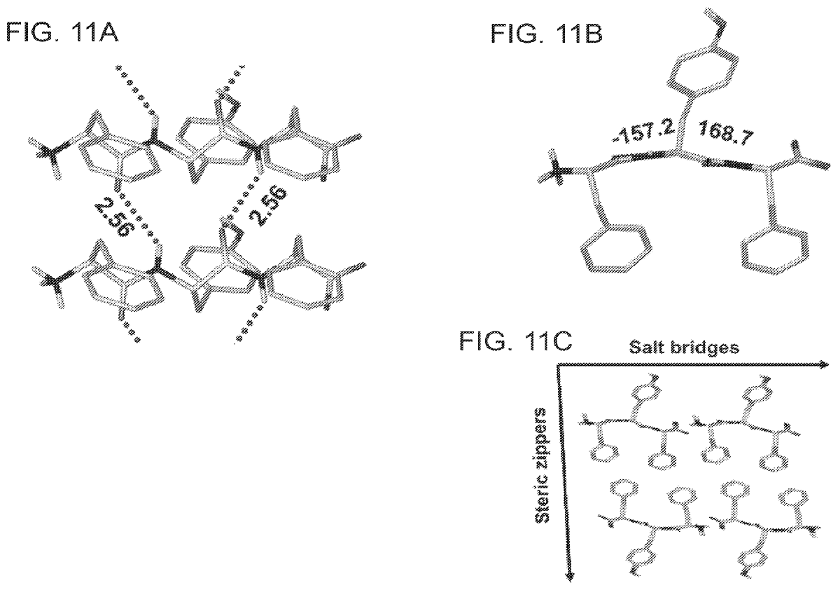

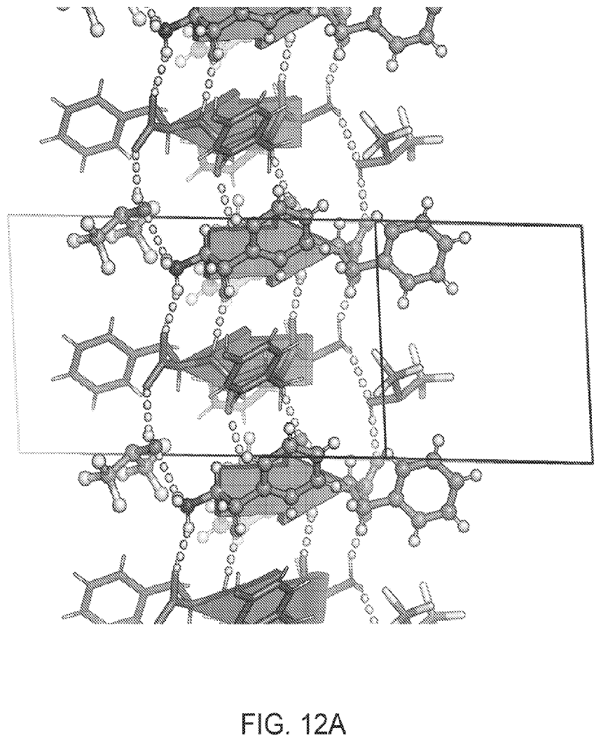

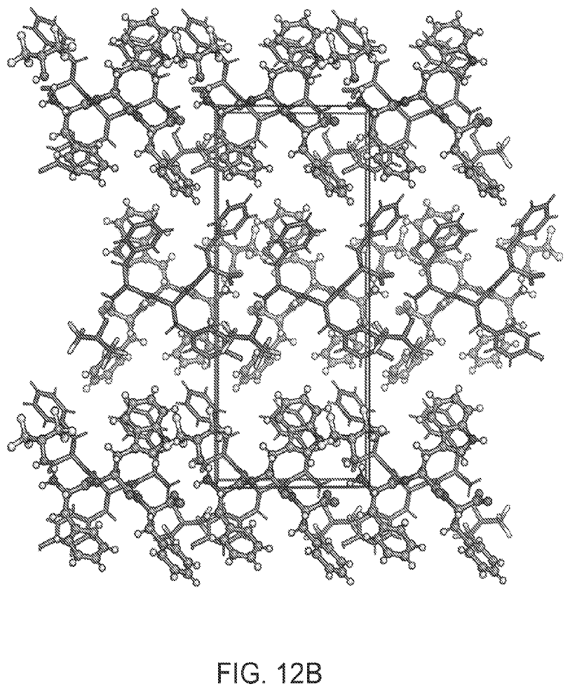

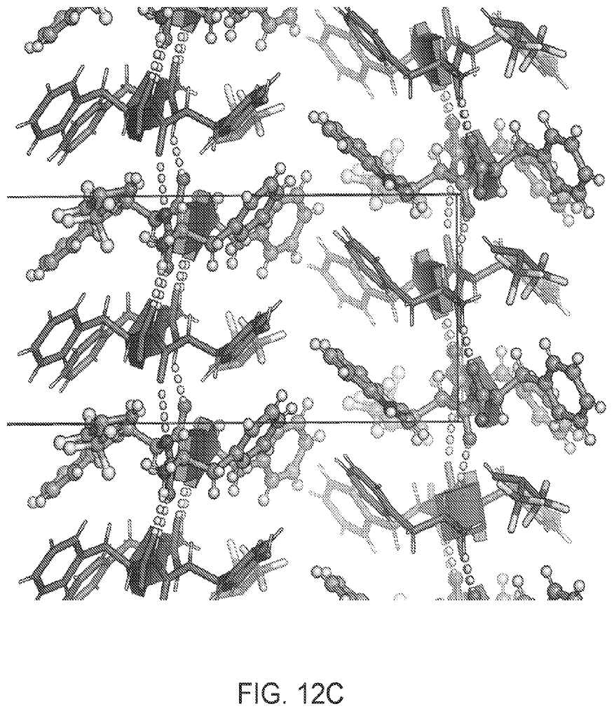

As part of this study, crystals of the enantiopure FYF tripeptide were also obtained. Whereas those crystals proved to be too small for conventional X-ray diffraction methods, micro electron diffraction (micro-ED) permitted successful structure solution and refinement (FIG. 11). Intriguingly, [FYF:FYF]n forms parallel (pleated) β-sheets, distinct from the three rippled β-sheet systems discussed above that are all antiparallel. The H-bonds connecting the FYF monomers in the fibril are measured as 2.56 Å (FIG. 11A). The torsional angles for the central residue of FYF were measured as φ=−157.2° and ψ=168.7° (FIG. 11 B), both substantially wider than the corresponding Ramachandran angles associated with FYF in the rippled antiparallel [FYF:fyf]n and [FWF:fyf]n β-sheets. This difference is likely a consequence of the distinct architecture and associated packing forces. Similar to the rippled systems, the pleated parallel [FYF:FYF]n columns are mated via steric zippers into fibrils that associate laterally through salt bridges (FIG. 11C).

An interesting parallel is noted with a recent study of racemic Aβ40.³¹ Whereas enantiopure Aβ40 is known to form parallel in-register pleated β-sheets, its racemic counterpart forms antiparallel rippled β-sheets, suggesting there may be an intrinsic preference for the rippled β-sheet to adopt the antiparallel orientation. Similar observations were made for the racemic aggregated FFF:fff system,³³ the racemic aggregated KLVFFAE:klvffae system,³ and for polyglycine I.²¹⁻²³ However, the preference is not absolute as the MAX1/DMAX hairpin system studied by the Schneider lab did form parallel rippled β-sheets.¹ Inter-sheet side-chain interactions may be another factor that could be explored to fine-tune rippled β-sheet architectures in the future.^{1, 36}

In conclusion, presented herein are the crystal structures of three distinct periodic rippled β-sheet layers, two of which are racemic (i.e., [FYF:fyf]n and [FWF:fwf]n), and one that is quasi-racemic (i.e., [FWF:fyf]n). The layer coordinates are in good overall agreement with the predictions of Pauling and Corey. The individual rippled β-sheet columns are mated via steric zippers into rippled β-sheet fibrils, which associate laterally via salt bridges. All three rippled β-sheet fibrils adopt the antiparallel arrangement, distinct from the pleated, homochiral [FYF:FYF]n that forms parallel β-sheet fibrils. The rippled β-sheet fibril is a novel supramolecular architecture which is expected to allow rational design of unique functional peptide materials.

### REFERENCES FOR EXAMPLE 2

1. K. Nagy-Smith, P. J. Beltramo, E. Moore, R. Tycko, E. M. Furst and J. P. Schneider, ACS Cent. Sci., 2017, 3, 586-597.
2. K. J. Nagy, M. C. Giano, A. Jin, D. J. Pochan and J. P. Schneider, J. Am. Chem. Soc., 2011, 133, 14975-14977.
3. J. M. Urban, J. Ho, G. Piester, R. Fu and B. L. Nilsson, Molecules, 2019, 24, 1983.
4. R. J. Swanekamp, J. J. Welch and B. L. Nilsson, Chem. Commun., 2014, 50, 10133-10136.
5. R. J. Swanekamp, J. T. M. DiMaio, C. J. Bowerman and B. L. Nilsson, J. Am. Chem. Soc., 2012, 134, 5556-5559.
6. K. M. Wong, A. S. Robang, A. H. Lint, Y. Wang, X. Dong, X. Xiao, D. T. Seroski, R. Liu, Q. Shao, G. A. Hudalla, C. K. Hall and A. K. Paravastu, J. Phys. Chem. B, 2021, 125, 13599-13609.
7. A. R. Foley, G. P. Roseman, K. Chan, A. Smart, T. S. Finn, K. Yang, R. S. Lokey, G. L. Millhauser and J. A. Raskatov, Proc. Natl. Acad. Sci. USA, 2020, 117, 28625-28631.
8. S. Dutta, T. S. Finn, A. J. Kuhn, B. Abrams and J. Raskatov, ChemBioChem, 2019, 20, 1023-1026.
9. A. R. Foley, T. S. Finn, T. Kung, A. Hatami, H.-W. Lee, M. Jia, M. Rolandi and J. A. Raskatov, ACS Chem. Neurosci., 2019, 10, 3880-3887.
10. C. J. A. Warner, S. Dutta, A. R. Foley, E. Chen, D. S. Kliger and J. A. Raskatov, Chirality, 2017, 29, 5-9.
11. S. Dutta, A. R. Foley, C. J. A. Warner, X. Zhang, M. Rolandi, B. Abrams and J. A. Raskatov, Angew. Chem. Int. Ed., 2017, 56, 11506-11510.
12. A. M. Garcia, C. Giorgiutti, Y. El Khoury, V. Bauer, C. Spiegelhalter, E. Leize-Wagner, P. Hellwig, N. Potier and V. Torbeev, Chem. Eur. J., 2020, 26, 9889-9899.
13. M. Avital-Shmilovici, J. Whittaker, M. A. Weiss and S. B. H. Kent, J. Biol. Chem., 2014, 289, 23683-23692.
14. T. O. Yeates and S. B. Kent, Annu. Rev. Biophys., 2012, 41, 41-61.
15. M. Avital-Shmilovici, K. Mandal, Z. P. Gates, N. B. Phillips, M. A. Weiss and S. B. H. Kent, J. Am. Chem. Soc., 2013, 135, 3173-3185.
16. R. D. Bunker, K. Mandal, G. Bashiri, J. J. Chaston, B. L. Pentelute, J. S. Lott, S. B. H. Kent and E. N. Baker, Proc. Natl. Acad. Sci. USA, 2015, 112, 4310-4315.
17. B. L. Pentelute, K. Mandal, Z. P. Gates, M. R. Sawaya, T. O. Yeates and S. B. H. Kent, Chem. Commun., 2010, 46, 8174-8176.
18. D. E. Mortenson, K. A. Satyshur, I. A. Guzei, K. T. Forest and S. H. Gellman, J. Am. Chem. Soc., 2012, 134, 2473-2476.
19. L. Pauling and R. B. Corey, Proc. Natl. Acad. Sci. USA, 1953, 39, 253-256.
20. L. Pauling and R. B. Corey, Proc. Natl. Acad. Sci. USA, 1951, 37, 729-740.
21. B. Lotz, J. Mol. Biol., 1974, 87, 169-180.
22. F. Colonna-Cesari, S. Premilat and B. Lotz, J. Mol. Biol., 1974, 87, 181-191.
23. W. H. Moore and S. Krimm, Biopolymers, 1976, 15, 2439-2464.
24. B. Lotz, ChemBioChem, 2022, 23, e202100658.
25. D. M. Chung and J. S. Nowick, J. Am. Chem. Soc., 2004, 126, 3062-3063.
26. J.-H. Fuhrhop, M. Krull and G. Buldt, Angew. Chem. Int. Ed., 1987, 26, 699-700.
27. I. Weissbuch, R. A. Illos, G. Bolbach and M. Lahav, Acc. Chem. Res., 2009, 42, 1128-1140.
28. J. A. Raskatov, ChemBioChem, 2020, 21, 2945-2949.
29. J. A. Raskatov, Biopolymers, 2020, DOI: 10.1002/bip.23391, e23391.
30. S. Dutta, A. Rodriguez Foley, A. Kuhn, B. Abrams, H.-W. Lee and J. A. Raskatov, Peptide Sci., 2019, 111, e24139.
31. J. A. Raskatov, A. R. Foley, J. M. Louis, W. M. Yau and R. Tycko, J. Am. Chem. Soc., 2021, 143, 13299-13313.
32. J. A. Raskatov, J. P. Schneider and B. L. Nilsson, Acc. Chem. Res., 2021, 54, 2488-2501.
33. A. J. Kuhn, B. Ehlke, T. C. Johnstone, S. R. J. Oliver and J. A. Raskatov, Chem. Sci., 2022, 13, 671-680.
34. M. R. Sawaya, S. Sambashivan, R. Nelson, M. I. Ivanova, S. A. Sievers, M. I. Apostol, M. J. Thompson, M. Balbirnie, J. J. W. Wiltzius, H. T. McFarlane, A. O. Madsen, C. Riekel and D. Eisenberg, Nature, 2007, 447, 453-457.
35. K. A. Murray, D. Evans, M. P. Hughes, M. R. Sawaya, C. J. Hu, K. N. Houk and D. Eisenberg, ACS Nano, 2022, 16, 2154-2163.
36. V. Torbeev, M. Grogg, J. Ruiz, R. Boehringer, A. Schirer, P. Hellwig, G. Jeschke and D. Hilvert, J. Peptide Sci., 2016, 22, 290-304.

### Materials and Methods for Example 2

### Peptide Synthesis and Crystallization

All peptides were synthesized on pre-loaded Wang resins by standard Fmoc based, solid-phase peptide chemistry. All syntheses were performed manually at 0.2 mM scale relative to resin loading. The peptides were cleaved and deprotected with a mixture consisting of trifluoroacetic acid (10 mL), tri-isopropylsilane (1 mL), and liquefied phenol (0.5 mL). The cleavage solution was added to the resins and agitated for 2 h. The solution was then evaporated to 2 mL under nitrogen gas, and the peptides precipitated with cold diethyl ether and centrifuged at 6000 rpm. The peptide pellet was washed with cold diethyl ether, dried, dissolved in 1:1 acetonitrile:water, flash frozen in liquid nitrogen, and lyophilized. No further purification was performed prior to crystallization.

### Crystallization and Crystal Structural Determination

[FYF:fyf]n

Solutions of the L-FYF and D-fyf peptides were prepared separately by dissolving 6 mg of each individual peptide in 300 μL of hexafluoroisopropanol. The resulting solutions were combined. Nanopure water (3 mL) was subsequently added. Colorless needles formed upon leaving the solution standing overnight. The needles were approximately 5 microns thick, making them suitable for X-ray diffraction at microfocal beamline 24-ID-E of the Advanced Photon Source located at Argonne National Laboratory. Crystals were cooled to a temperature of 100 K. Diffraction data from three crystals were indexed, integrated, scaled, and merged using the programs XDS and XSCALE. An atomic model was obtained by direct methods using the program ShelxD. The model was refined using the program SHELXL, and manually edited using the graphics program Coot. Some of the structure illustrations were created using PyMOL.

Data collection and refinement statistics
for the structure of racemic FYF:fyf

| Peptide Sequence | FYF:fyf |
|---|---|
| X-ray Source | APS 24-ID-E |
| Space group | C2/c |
| Resolution (Å) | 1.10 (1.13-1.10)* |
| Unit cell lengths: a, b, c (Å) | 22.02, 9.57, 25.84 |
| Unit cell angles: α, β, γ (°) | 90.0, 102.35, 90.0 |
| Measured reflections | 30513 (670) |
| Unique reflections | 1905 (81) |
| Overall completeness (%) | 91.8 (57.0) |
| Overall redundancy | 16.0 (8.2) |
| Overall R_{merge} | 0.082 (0.155) |
| CC_{1/2} | 99.9 (98.6) |
| Overall I/δ | 25.5 (10.7) |
| Refinement | |
| R_{work}/R_{free} | 0.055/0.066 |
| RMSD bond length (Å) | 0.015 |
| RMSD angle (°) | 1.4 |
| Number of peptide atoms (incl. H) | 64 |
| Number of water molecules | 3 |
| Number of other solvent atoms | 0 |
| Average B-factor of peptide (Å²) | 5.1 |
| Average B-factor of water (Å²) | 16.3 |
| Average B-factor other solvent (Å²) | N/A |
| PDB ID code | tba |

[FWF:fwf]n

Solutions of the L-FWF and D-fwf peptides were prepared separately by dissolving 1 mg of each individual peptide in a solution of trifluoroethanol in Nanopure water (1:10, 1.1 mL). The resulting solutions were combined. Colorless needles formed upon leaving the solution standing for 3 weeks. A suitable colorless needle with dimensions of 0.26×0.04×0.03 mm³ was used for single-crystal X-ray diffraction data collection at 100 K on a Rigaku XtaLAB Synergy-S diffractometer using Cu Kα radiation (λ=1.54 Å). Data collection, processing and reduction were performed with CrysAlisPro. After face indexing, a numerical absorption correction was applied using gaussian integration. An empirical absorption correction using spherical harmonics was applied with the SCALE3 ABSPACK scaling algorithm. The structure was solved by intrinsic phasing using ShelXT and refined with ShelXL using Olex2.⁶ All non-hydrogen atoms were refined anisotropically using standard procedures.⁷ Atomic displacement parameters for hydrogen atoms were fixed to 1.2×Uᵢₛₒ of the atoms to which they are attached. All hydrogen atoms were placed at geometrically calculated positions and refined using a riding model. For the ammonium groups, a modified riding model was used in which H atoms are allowed to rotate about the C—N bond. The N—H distances in the ammonium and amide/indole groups were constrained to 0.91 Å and 0.88 Å, respectively. The O—H distance of the carboxylic acid was constrained to 0.84 Å. All H-atom positions were consistent with the hydrogen-bonding patterns dictated by the heavier atoms. A pocket of disordered solvent molecules (66 e⁻ in 176 Å³; consistent with 1 trifluoroethanol and 1 water) could not be satisfactorily refined. The contribution to this disordered solvent to the observed structure factors was masked using Olex2.

Data collection and Refinement statistics for racemic FWF:fwf

| | |
|---|---|
| T (K) | 100(2) |
| λ (Å) | 1.54184 |
| Crystal System | Monoclinic |
| Space group | P2₁/c |
| a (Å) | 9.5344(5) |
| b (Å) | 23.7071(16) |
| c (Å) | 24.8206(12) |
| β (°) | 99.040(5) |
| Volume (Å³) | 5540.6(5) |
| Z | 8 |
| ρ_{calc} (Mg/m³) | 1.197 |
| Size (mm³) | 0.26 × 0.04 × 0.03 |
| θ range (°) | 2.593-67.078 |
| Total data | 42092 |
| Unique data | 9787 |
| Parameters | 670 |
| Completeness | 98.7% |
| Rᵢₙₜ | 8.73% |
| R₁ (I > 2σ) | 8.17% |
| R₁ (all data) | 14.11% |
| wR₂ (I > 2σ) | 22.23% |
| wR₂ (all data) | 26.16% |
| S | 1.049 |
| Min, max (e Å⁻³) | −0.466, 0.568 |
| Deposition number | CCDC 2168013 |

[FWF:fyf]n

Solutions of the L-FWF and D-fyf peptides were prepared separately by dissolving 1.5 mg of each individual peptide in 50 μl and 100 μl of hexafluoroisopropanol, respectively. The resulting solutions were combined. Nanopure water (1 mL) was subsequently added. Colorless needles formed upon leaving the solution standing for several days.

| Peptide Sequence | FWF:fyf |
|---|---|
| X-ray Source | APS 24-ID-E |
| Space group | P2₁ |
| Resolution (Å) | 1.10 (1.13-1.10)* |
| Unit cell lengths: a, b, c (Å) | 9.66, 26.26, 11.63 |
| Unit cell angles: α, β, γ (°) | 90.0, 95.12, 90.0 |
| Measured reflections | 13143 (308) |
| Unique reflections | 2242 (113) |
| Overall completeness (%) | 93.8 (66.1) |
| Overall redundancy | 5.9 (2.7) |
| Overall R_{merge} | 0.076 (0.255) |
| CC_{1/2} | 99.8 (94.3) |
| Overall I/δ | 13.0 (3.6) |
| Refinement | |
| R_{work}/R_{free} | 0.092/0.127 |
| RMSD bond length (Å) | 0.017 |
| RMSD angle (°) | 2.7 |
| Number of peptide atoms (incl. H) | 168 |
| Number of water molecules | 1 |
| Number of other solvent atoms | 22 |
| Average B-factor of peptide (Å²) | 12.9 |
| Average B-factor of water (Å²) | 14.8 |
| Average B-factor other solvent (Å²) | 14.9 |
| PDB ID code | tba |

### [FYF:FYF]n

A solution of the L-FYF peptide was prepared by dissolving 5 mg of the peptide in 3 mL of nanopure water (3 mL). Colorless needles, suitable for micro-electron diffraction formed after several hours. 3 μL was deposited directly from the batch suspension on to an ultrathin carbon/lacey TEM grid using the dropcast technique. The sample was inserted at room temperature in a specialized sample holder designed for cryo-electron microscopy, and subsequently cooled to −177° C. Electron diffraction data was collected at a Tecnai F30 TEM operating at 300 kV with a flux density of approximately 0.0192 electrons per square Angstrom per frame, such that the total accumulated dose by a crystal during a typical tilt series was approximately 8.04 MGy. Single crystals were located on the grid and centered in the microscope's selected area aperture, and continuous rotation diffraction tilt series were collected for each. Data was processed in XDS with a high-resolution limit of 0.9 Angstroms, and intensities from two crystals were merged in space group C2 to give an overall completeness of 71%, which was sufficient to resolve a structure solution by direct methods using SHELXD. Merging data from up to three additional crystals did not enhance the completeness of the data beyond 75%, at the expense of higher R_{merge} values.

Structure refinement was performed in PHENIX, where a final R_{work}/R_{free} of 16.85% and 17.08% respectively were reached after the addition of hydrogen atoms and anisotropic treatment of non-H atoms using PHENIX's TLS (Translation/Libration/Screw) routine. Individual anisotropic refinement of B-factors was also attempted in PHENIX, but this yielded a greater gap between R_{work} and R_{free}, without any improvement to R_{work} over the TLS result. As such, the refined structure reported is the iteration refined using TLS.

Data collection and Refinement statistics for the structure of homochiral FYF

| Peptide Sequence | FYF Crystal 1 | FYF Crystal 2 | FYF merge |
|---|---|---|---|
| Electron Source | Tecnai F30 TEM | Tecnai F30 TEM | |
| Space group | C2 | C2 | C2 |
| Resolution (Å) | 0.90 (1.00-0.90)* | 0.90 (1.00-0.90) | 0.90 (1.00-0.90) |
| Unit cell lengths: a, b, c (Å) | 23.14, 4.84, 19.79 | 23.08, 4.84, 19.55 | 23.14, 4.84, 19.79 |
| Unit cell angles: α, β, γ (°) | 90.00, 107.05, 90.00 | 90.00, 106.62, 90.00 | 90.00, 107.05, 90.00 |
| Measured reflections | 3015 (801) | 2956 (835) | 5980 (1639) |
| Unique reflections | 1097 (288) | 987 (275) | 1237 (332) |
| Overall completeness (%) | 63.0 (62.6) | 57.3 (60.2) | 71.0 (72.2) |
| Overall redundancy | 2.7 (2.8) | 3.0 (3.0) | 4.8 (2.7) |
| Overall R_{merge} | 0.106 (0.320) | 0.092 (0.206) | 0.113 (0.323) |
| CC_{1/2} | 99.1 (60.6) | 99.1 (92.3) | 99.1 (64.3) |
| Overall I/δ | 6.7 (3.5) | 8.1 (4.9) | 8.6 (5.0) |

| | |
|---|---|
| Refinement | |
| R_{work}/R_{free} | 0.169/0.185 |
| RMSD bond length (Å) | 0.020 |
| RMSD angle (°) | 1.25 |
| Number of peptide atoms (incl. H) | 64 |
| Number of water molecules | 0 |
| Number of other solvent atoms | 0 |
| Average B-factor of peptide (Å²) | 3.3 |
| Average B-factor of water (Å²) | N/A |
| Average B-factor other solvent (Å²) | N/A |
| PDB ID code | tba |

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A rippled antiparallel cross-β dimer comprising (L,L,L)-(FX₁F)ₖ dimerized with (D,D,D)-(FX₂F)ₖ, wherein X₁ and X₂ are independently selected from any amino acid, and wherein k is an integer of 1 or greater.

2. The rippled antiparallel cross-β dimer of claim 1, wherein X₁ and X₂ are independently selected from the group consisting of: F, Y, and W.

3. The rippled antiparallel cross-β dimer of claim 1 or claim 2, wherein X₁ and X₂ are the same.

4. The rippled antiparallel cross-β dimer of claim 1 or claim 2, wherein X₁ and X₂ are different.

5. The rippled antiparallel cross-β dimer of any one of claims 1 to 4, wherein the dimer is water-soluble.

6. The rippled antiparallel cross-β dimer of claim 5, wherein the N-terminus of the (L,L,L)-(FX₁F)ₖ monomer comprises a free amine or a free carboxylate.

7. The rippled antiparallel cross-β dimer of claim 5 or claim 6, wherein the N-terminus of the (D,D,D)-(FX₂F)ₖ monomer comprises a free amine or a free carboxylate.

8. A rippled β-sheet fibril comprising a plurality of the rippled antiparallel cross-β dimers of any one of claims 1 to 7.

9. A material comprising a plurality of the rippled antiparallel cross-β dimers of any one of claims 1 to 7.

10. A material comprising the rippled β-sheet fibril of claim 8.

11. The material of claim 9 or claim 10, wherein the material comprises the plurality of rippled antiparallel cross-β dimers held together by a combination of interdimer hydrogen bonds, ionic interactions, and van der Waals interactions.

12. A composition comprising the material of any one of claims 9 to 11.

13. The composition of claim 12, wherein the dimer or material is present in a liquid medium.

14. The composition of claim 13, wherein the liquid medium is an aqueous liquid medium.

15. A method comprising:
producing a polypeptide comprising, consisting essentially of, or consisting of, (L,L,L)-(FX₁F)ₖ; and
producing a polypeptide comprising, consisting essentially of, or consisting of, (D,D,D)-(FX₂F)k,
wherein (L,L,L)-(FX₁F)ₖ and (D,D,D)-(FX₂F)ₖ are as defined in any one of claims 1 to 7.

16. The method according to claim 15, wherein the polypeptides are produced by chemical synthesis.

17. The method according to claim 16, wherein the chemical synthesis is by solid-phase polypeptide synthesis.

18. The method according to claim 17, wherein the solid-phase polypeptide synthesis is Fmoc-based solid-phase peptide synthesis.

19. The method according to any one of claims 15 to 18, further comprising purifying the produced (L,L,L)-(FX₁F)ₖ and (D,D,D)-(FX₂F)ₖ.

20. The method according to any one of claims 15 to 19, further comprising combining the produced (L,L,L)-(FX₁F)ₖ and (D,D,D)-(FX₂F)ₖ into a racemic mixture.

21. A method comprising:
combining (L,L,L)-(FX₁F)ₖ and (D,D,D)-(FX₂F)ₖ in a mixture under conditions in which rippled antiparallel cross-β dimers comprising (L,L,L)-(FX₁F)ₖ and (D,D,D)-(FX₂F)ₖ are formed,
wherein (L,L,L)-(FX₁F)ₖ and (D,D,D)-(FX₂F)ₖ are as defined in any one of claims 1 to 7.

22. The method according to claim 21, wherein a rippled β-sheet fibril comprising the rippled antiparallel cross-β dimers is formed.
